(19) United States
(12) Patent Application Publication
(10) Pub. No.: US 2026/0337184 A1
(43) Pub. Date: Oct. 29, 2026

(54) NOVEL SUBSTITUTED 2-CARBONYL-BENZOTHIOPHENE-6-CARBOXYLIC ACID DERIVATIVES, PROCESSES FOR THEIR PREPARATION AND THERAPEUTIC USES THEREOF

(71) Applicant: SANOFI, Paris (FR)

(72) Inventor: Youssef El-Ahmad, Paris (FR)

(21) Appl. No.: 19/105,437

(22) PCT Filed: Aug. 24, 2023
(86) PCT No.: PCT/EP2023/073209

§ 371 Date: Feb. 21, 2025

(30) Foreign Application Priority Data
Aug. 25, 2022 (EP) 22306253.0

(51) Int. Cl. C07D 409/12; A61K 31/397; A61K 31/4025; A61P 35/00

(52) U.S. Cl. CPC C07D 409/12; A61K 31/397; A61K 31/4025; A61P 35/00

(57) ABSTRACT

Disclosed herein are compounds of the formula (I), or pharmaceutically acceptable salts thereof, wherein R1 and R2 independently represent a hydrogen atom or a deuterium atom; R3′ and R3″ independently represent a hydrogen atom, a methyl group, a methoxy group, a chlorine atom, a fluorine atom, or a cyano group; R4 represents a hydrogen atom or a fluorine atom; R5 and R5′ independently represent a hydrogen atom or a fluorine atom; (aa) represents —CH₂—, —CH═, —CR8=, —O— or —NH—, wherein R8 represents a fluorine atom or a (C₁-C₃)alkyl group; represents a single bond or a double bond; X represents —CH═, —N═ or —CR″═, wherein R″ represents a (C₁-C₃)alkyl group or a halogen atom, such as a fluorine or a chlorine atom, a cyano group, or a (C₁-C₃)fluoroalkyl group, such as a trifluoromethyl; R7 independently represents a (C₁-C₃)alkyl group, such as a methyl group, a halogen atom, such as a fluorine atom, a cyano group, or a (C₁-C₃)fluoroalkyl group, such as a trifluoromethyl; and R6 represents an optionally substituted mono or bicyclic ring selected from a phenyl group, a 5- or 6-membered monocyclic heteroaryl group, and a 7-, 8-, 9- or 10-membered bicyclic heterocycle group. Further disclosed are process for preparing the same, pharmaceutical compositions comprising them as well as said compounds of formula (I) for use as an inhibitor and degrader of estrogen receptors, in particular in the treatment of ovulatory dysfunction, cancer, endometriosis, osteoporosis, benign prostatic hypertrophy or inflammation.

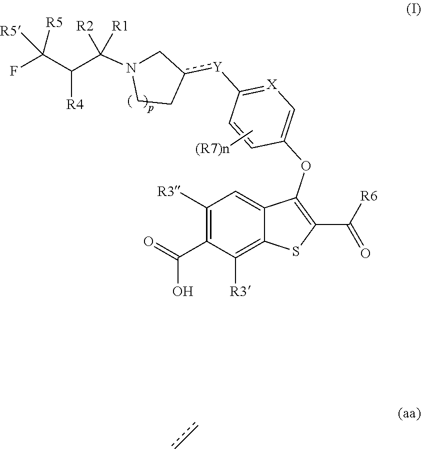

Disclosed herein are novel substituted 2-carbonyl-benzothiophene-6-carboxylic acid derivatives, the processes for their preparation, as well as the therapeutic uses thereof, in particular as anticancer agents via selective antagonism and degradation of estrogen receptors.

The Estrogen Receptors (ER) belong to the steroid/nuclear receptor superfamily involved in the regulation of eukaryotic gene expression, cellular proliferation and in target tissues. ERs are in two forms: the estrogen receptor alpha (ERα) and the estrogen receptor beta (ERβ) respectively encoded by the ESR1 and the ESR2 genes. ERα and ERβ are ligand-activated transcription factors which are activated by the hormone estrogen (the most potent estrogen produced in the body is 17β-estradiol). In the absence of hormone, ERs are largely located in the cytosol of the cell. When the hormone estrogen binds to ERs, ERs migrate from the cytosol to the nucleus of the cell, form dimers and then bind to specific genomic sequences called Estrogen Response Elements (ERE). The DNA/ER complex interacts with co-regulators to modulate the transcription of target genes.

ERα is mainly expressed in reproductive tissues such as uterus, ovary, breast, bone and white adipose tissue. Abnormal ERα signaling leads to development of a variety of diseases, such as cancers, metabolic and cardiovascular diseases, neurodegenerative diseases, inflammation diseases and osteoporosis.

ERα is expressed in not more than 10% of normal breast epithelium but approximately 50-80% of breast tumors. Such breast tumors with high level of ERα are classified as ERα-positive breast tumors. The etiological role of estrogen in breast cancer is well established and modulation of ERα signaling remains the mainstay of breast cancer treatment for the majority ERα-positive breast tumors. Currently, several strategies for inhibiting the estrogen axis in breast cancer exist, including: 1-blocking estrogen synthesis by aromatase inhibitors that are used to treat early and advanced ERα-positive breast cancer patients; 2-antagonizing estrogen ligand binding to ERα by tamoxifen which is used to treat ERα-positive breast cancer patients in both pre- and post-menopausal setting; 3-antagonizing and downregulating ERα levels by fulvestrant, which is used to treat breast cancer in patients that have progressed despite endocrine therapies such as tamoxifen or aromatase inhibitors.

Although these endocrine therapies have contributed enormously to reduction in breast cancer development, about more than one-third of ERα-positive patients display de novo resistance or develop resistance over time to such existing therapies. Several mechanisms have been described to explain resistance to such hormone therapies. For example, hypersensitivity of ERα to low estrogen level in treatment with aromatase inhibitors, the switch of tamoxifen effects from antagonist to agonist effects in tamoxifen treatments or multiple growth factor receptor signaling pathways. Acquired mutations in ERα occurring after initiation of hormone therapies may also play a role in treatment failure and cancer progression. Certain mutations in ERα, particularly those identified in the Ligand Binding Domain (LBD), result in the ability to bind to DNA in the absence of ligand and confer hormone independence in cells harboring such mutant receptors.

Most of the endocrine therapy resistance mechanisms identified rely on ERα-dependent activity. One of the new strategies to counterforce such resistance is to shut down the ERα signaling by removing ERα from the tumor cells using Selective Estrogen Receptors Degraders (SERDs). Clinical and preclinical data showed that a significant number of the resistance pathways can be circumvented by the use of SERDs.

There is still a need to provide SERDs with good degradation efficacy.

Documents WO2017/140669 and WO2018/091153 disclose some substituted 6,7-dihydro-5H-benzo[7]annulene compounds and substituted N-(3-fluoropropyl)-pyrrolidine derivatives useful as SERDs.

The inventors have now found novel compounds able to selectively antagonize and degrade the estrogen receptors (SERDs compounds), for use in cancer treatment.

Described herein are compounds of the formula (I), or pharmaceutically acceptable salts thereof:

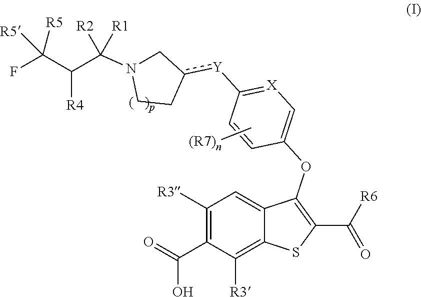

wherein:
   R1 and R2 independently represent a hydrogen atom or a deuterium atom;
   R3′ and R3″ independently represent a hydrogen atom, a methyl group, a methoxy group, a chlorine atom, a fluorine atom, or a cyano group;
   R4 represents a hydrogen atom or a fluorine atom;
   R5 and R5′ independently represent a hydrogen atom or a fluorine atom;
   Y represents —CH₂—, —CH═, —CR8=, —O— or —NH—, wherein R8 represents a fluorine atom or a (C₁-C₃)alkyl group;

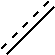

represents a single bond or a double bond;
p is 0 or 1;
X represents —CH═, —N═ or —CR″═, wherein R″ represents a (C₁-C₃)alkyl group or a halogen atom, such as a fluorine or a chlorine atom, a cyano group, or a (C₁-C₃)fluoroalkyl group, such as a trifluoromethyl;
R7 independently represents a (C₁-C₃)alkyl group, such as a methyl group, a halogen atom, such as a fluorine atom, a cyano group, or a (C₁-C₃)fluoroalkyl group, such as a trifluoromethyl;
n is 0, 1 or 2; and
R6 represents a mono or bicyclic ring selected from
   a phenyl group,
   a 5- or 6-membered monocyclic heteroaryl group, and
   a 7-, 8-, 9- or 10-membered bicyclic heterocycle group, said mono or bicyclic ring being optionally substituted by 1, 2, 3 or 4 groups selected from a halogen atom, a hydroxyl group, a (C₁-C₆)alkyl group optionally substituted with a cyano group or a —OH group, a (C₃-C₆)cycloalkyl group, a (C₁-C₆)fluoroalkyl group, a —CN group, a (C₁-C₆)alkoxy group, a (C₁-C₆) fluoroalkoxy group, a trifluoromethylsulfonyl group, a (C₁-C₄)alkylthio group, a (C₁-C₄)fluoroalkylthio group and a (C₁-C₄)alkylsulfonyl group.

Disclosed herein are compounds of the formula (I), or pharmaceutically acceptable salts thereof:

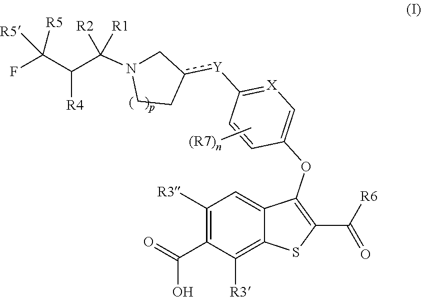

wherein:
   R1 and R2 independently represent a hydrogen atom or a deuterium atom;
   R3′ and R3″ independently represent a hydrogen atom, a methyl group, a methoxy group, a chlorine atom, a fluorine atom, or a cyano group;
   R4 represents a hydrogen atom or a fluorine atom;
   R5 and R5′ independently represent a hydrogen atom or a fluorine atom;
   Y represents —CH₂—, —CH═, —CR8=, —O— or —NH—, wherein R8 represents a fluorine atom or a (C₁-C₃)alkyl group;

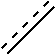

represents a single bond or a double bond;
p is 0 or 1;
X represents —CH═, —N═ or —CR″═, wherein R″ represents a (C₁-C₃)alkyl group or a halogen atom, such as a fluorine or a chlorine atom, a cyano group, or a (C₁-C₃)fluoroalkyl group, such as a trifluoromethyl;
R7 independently represents a (C₁-C₃)alkyl group, such as a methyl group, a halogen atom, such as a fluorine atom, a cyano group, or a (C₁-C₃)fluoroalkyl group, such as a trifluoromethyl;
n is 0, 1 or 2; and
R6 represents a mono or bicyclic ring selected from
   a phenyl group,
   a 5- or 6-membered monocyclic heteroaryl group, and
   a 7-, 8-, 9- or 10-membered bicyclic heterocycle group, said mono or bicyclic ring being optionally substituted by 1, 2, 3 or 4 groups selected from a halogen atom, a hydroxyl group, a (C₁-C₆)alkyl group optionally substituted with a cyano group or a —OH group, a (C₃-C₆)cycloalkyl group, a (C₁-C₆)fluoroalkyl group, a —CN group, a (C₁-C₆)alkoxy group, a (C₁-C₆) fluoroalkoxy group, a trifluoromethylsulfonyl group, a (C₁-C₄)alkylthio group, a (C₁-C₄)fluoroalkylthio group and a (C₁-C₄)alkylsulfonyl group, provided that the compounds with p=1 and Y is —O— are excluded.

The compounds of formula (I) can contain one or more asymmetric carbon atoms. They may therefore exist in the form of enantiomers.

The compounds of formula (I) may be present as well under tautomer forms.

The compounds of formula (I) may exist in the form of bases, acids, zwitterion or of addition salts with acids or bases. Hence, herein are provided compounds of formula (I) or pharmaceutically acceptable salts thereof.

These salts may be prepared with pharmaceutically acceptable acids or bases, although the salts of other acids or bases useful, for example, for purifying or isolating the compounds of formula (I) are also provided.

Among suitable salts of the compounds of formula (I), hydrochloride may be cited.

As used herein, the terms below have the following definitions unless otherwise mentioned throughout the instant specification:
a halogen atom: a fluorine, a chlorine, a bromine or an iodine atom, and in particular a fluorine and a chlorine atom;
an alkyl group: a linear or branched saturated hydrocarbon-based aliphatic group comprising, unless otherwise mentioned, from 1 to 6 carbon atoms (noted “(C1-C6)-alkyl”). By way of examples, mention may be made of, but not limited to: methyl, ethyl, propyl, isopropyl, butyl, isobutyl, sec-butyl, tert-butyl, pentyl, isopentyl, hexyl and isohexyl groups, and the like;
a cycloalkyl group: a monocyclic alkyl group comprising, unless otherwise mentioned, from 3 to 7 carbon atoms, saturated or partially unsaturated and unsubstituted or substituted. By way of examples, mention may be made of, but not limited to: cyclopropyl, cyclobutyl, cyclopentyl, cyclobutenyl, cyclopentenyl, cyclohexyl, cyclohexenyl, cycloheptyl, cycloheptenyl, groups and the like, in particular a cyclopentyl, a cyclohexyl, a cycloheptyl, a cycloheptenyl, or a cyclohexenyl;
a heterocycloalkyl group: a 4 to 7-membered cycloalkyl group, saturated or partially unsaturated, comprising 1 to 2 heteroatoms independently selected from oxygen, nitrogen and sulfur, in particular being oxygen or nitrogen;
a monocyclic heterocycle group: a 3, 4, 5, 6 or 7 membered ring containing at least one heteroatom independently selected from the group consisting of O, N, and S where the ring is saturated or unsaturated, but not aromatic. A 3 or 4 membered ring may contain 1 heteroatom selected from the group consisting of O, N and S. A 5 membered ring may contain zero or one double bond and one, two or three heteroatoms selected from the group consisting of O, N and S. A 6 or 7 membered ring may contain zero, one or two double bonds and one, two or three heteroatoms selected from the group consisting of O, N and S;
a 7-, 8-, 9- or 10-membered bicyclic heterocycle group: a 7 to 10-membered bicyclic group, wherein a monocyclic heterocycloalkyl group is fused to either a phenyl group, a monocyclic cycloalkyl group, such as a (C₃-C₆)cycloalkyl group, a monocyclic cycloalkenyl group, a monocyclic heterocycloalkyl group, or a monocyclic heteroaryl group. The bicyclic heterocycle may be connected to the parent molecular moiety through any carbon atom or any nitrogen atom contained within the monocyclic or bicyclic heterocycle portion of the bicyclic ring system;
a fluoroalkyl group: an alkyl group as previously defined where the alkyl group is substituted with at least one fluorine atom. In other terms, at least one hydrogen atom of the alkyl group is replaced by a fluorine atom. By way of example, mention may be made of CH₂F, CHF₂, CH₂CHF₂, —CH₂CH₂F and the like. When all the hydrogen atoms belonging to the alkyl group are replaced by fluorine atoms, the fluoroalkyl group can be named perfluoroalkyl group. By way of example, mention may be made of trifluoromethyl group or trifluoroethyl group and the like;
an alkoxy group: an —O-alkyl group where the alkyl group is as previously defined. By way of examples, mention may be made of, but not limited to: methoxy, ethoxy, propoxy, isopropoxy, linear, secondary or tertiary butoxy, isobutoxy, pentoxy or hexoxy groups, and the like;
a fluoroalkoxy group: an —O-alkyl group where the alkyl group is as previously defined and where the alkyl group is substituted with at least one fluorine atom. In other terms, at least one hydrogen atom of the alkyl group is replaced by a fluorine atom. By way of example, mention may be made of —OCH₂F, —OCHF₂, —OCH₂CH₂F and the like. When all the hydrogen atoms belonging to the alkyl group are replaced by fluorine atoms, the fluoroalkoxy group can be named perfluoroalkoxy group. By way of example, mention may be made of trifluoromethoxy group and the like;
a (C₁-C₄)alkylthio group also named a (C₁-C₄)alkylsulfanyl group: a —S-alkyl group where the alkyl group is as previously defined. By way of examples, mention may be made of, but not limited to: methylthio, ethylthio, propylthio, isopropylthio, linear, secondary or tertiary butylthio, isobutylthio, and the like;
a (C₁-C₄)alkylsulfonyl group: a —SO₂-alkyl group where the alkyl group is as previously defined. By way of examples, mention may be made of, but not limited to —SO₂CH₃, —SO₂CH₂CH₃ and the like;
a (C₁-C₄)fluoroalkylthio group also named a (C₁-C₄)fluoroalkylsulfanyl group: a —S-fluoroalkyl group where the fluoroalkyl group is as previously defined. By way of examples, mention may be made of, but not limited to: fluoromethylthio, difluoromethylthio, trifluoromethylthio and the like;
a 5- or 6-membered monocyclic heteroaryl group: a cyclic 5 or 6-membered aromatic group. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl.

In another embodiment, in the compounds of formula (I) as defined above, R1 and R2 are a hydrogen atom.

In another embodiment, in the compounds of formula (I) as defined above, R3′ and R3″ both represent a hydrogen atom.

In another embodiment, in the compounds of formula (I) as defined above, R4 is a hydrogen atom.

In another embodiment, in the compounds of formula (I) as defined above, R5 and R5′ both represent a hydrogen atom.

In another embodiment, in the compounds of formula (I) as defined above, p is 0. In another embodiment, in the compounds of formula (I) as defined above, p is 1.

In another embodiment, in the compounds of formula (I) as defined above, Y is —CH₂—, —CH═, —O— or —NH—.

In another embodiment,

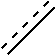

represents a single bond. In another embodiment,

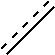

represents a double bond.

In another embodiment, in the compounds of formula (I) as defined above, n is 0.

In another embodiment, in the compounds of formula (I) as defined above, n is 0. In another embodiment, in the compounds of formula (I) as defined above, n is 1. In another embodiment, in the compounds of formula (I) as defined above, n is 2.

In another embodiment, in the compounds of formula (I) as defined above, X represents —CH═.

In another embodiment, in the compounds of formula (I) as defined above, R6 represents a phenyl group optionally substituted by 1, 2, 3 or 4 groups selected from a halogen atom, and a (C₁-C₆)alkyl group, more particularly optionally substituted by 2, 3 or 4 groups selected from a halogen, for example a fluorine atom, and a methyl group and even more particularly by one fluorine atom and by two methyl groups.

In another embodiment, Y is —CH₂—, —CH═ or —CR8=.

In an embodiment, X represents a carbon atom (i.e., a ═CH— group) in the compounds of formula (I). Accordingly, herein are described compounds of formula (II):

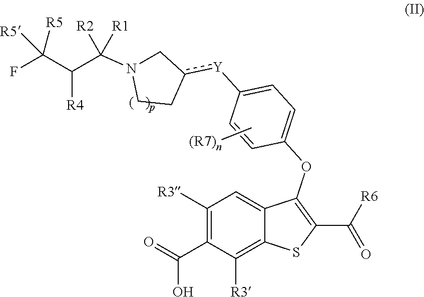

wherein R1, R2, R3′, R3″, R4, R5, R5′, R6, R7,

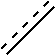

Y, n and p are as defined in formula (II) hereabove.

The embodiments as described above for compounds of formula (I) are also suitable for compounds of formula (II).

In another embodiment, in the compounds of formula (II) as defined above, R6 represents a phenyl group optionally substituted by 1, 2, 3 or 4 groups selected from a halogen atom, and a (C₁-C₆)alkyl group, more particularly optionally substituted by 2, 3 or 4 groups selected from a halogen, for example a fluorine atom, and a methyl group and even more particularly by one fluorine atom and by two methyl groups. Among the compounds of formula (I) described herein, mention may be made in particular of the following compounds or a pharmaceutically acceptable salt thereof, in particular hydrochloride salt thereof:
2-(4-fluoro-2,6-dimethylbenzoyl)-3-(4-((1-(3-fluoropropyl)azetidin-3-yl)amino)phenoxy)benzo[b]thiophene-6-carboxylic acid (1),
2-(4-fluoro-2,6-dimethylbenzoyl)-3-(4-((1-(3-fluoropropyl)azetidin-3-yl)oxy)phenoxy)benzo[b]thiophene-6-carboxylic acid (2),
2-(4-fluoro-2,6-dimethylbenzoyl)-3-(4-((1-(3-fluoropropyl)azetidin-3-yl)methyl)phenoxy)benzo[b]thiophene-6-carboxylic acid (3),
2-(4-fluoro-2,6-dimethylbenzoyl)-3-(4-((1-(3-fluoropropyl)azetidin-3-ylidene)methyl)phenoxy)benzo[b]thiophene-6-carboxylic acid (4),
(S)-2-(4-fluoro-2,6-dimethylbenzoyl)-3-(4-((1-(3-fluoropropyl)pyrrolidin-3-yl)oxy)phenoxy)benzo[b]thiophene-6-carboxylic acid (5), and
(S)-2-(4-fluoro-2,6-dimethylbenzoyl)-3-(4-((1-(3-fluoropropyl)pyrrolidin-3-yl)amino)phenoxy)benzo[b]thiophene-6-carboxylic acid (6).

Another embodiment is a compound selected from the above list, or a pharmaceutically acceptable salt thereof, for use in therapy, especially as an inhibitor and degrader of estrogen receptors.

Another embodiment is a compound selected from the above list, or a pharmaceutically acceptable salt thereof, for use in the treatment of cancer, especially breast cancer.

Another embodiment is a method of inhibiting and degrading estrogen receptors, comprising administering to a subject in need thereof, in particular a human, a therapeutically effective amount of a compound selected from the above list, or a pharmaceutically acceptable salt thereof.

Another embodiment is a method of treating ovulatory dysfunction, cancer, endometriosis, osteoporosis, benign prostatic hypertrophy or inflammation, comprising administering to a subject in need thereof, in particular a human, a therapeutically effective amount of a compound selected from the above list, or a pharmaceutically acceptable salt thereof.

Another embodiment is a method of treating cancer, comprising administering to a subject in need thereof, in particular a human, a therapeutically effective amount of a compound selected from the above list, or a pharmaceutically acceptable salt thereof.

Another embodiment is a pharmaceutical composition comprising as active principle an effective dose of a compound selected from the above list, or a pharmaceutically acceptable salt thereof, and also at least one pharmaceutically acceptable excipient.

The compounds of the formula (I) can be prepared by the following processes.

The compounds of the formula (I) and other related compounds having different substituents are synthesized using techniques and materials described below or otherwise known by the skilled person in the art. In addition, solvents, temperatures and other reaction conditions presented below may vary as deemed appropriate to the skilled person in the art. General below methods for the preparation of compounds of formula (I) optionally modified by the use of appropriate reagents and conditions for the introduction of the various moieties found in the formula (I) are described below.

The following abbreviations and empirical formulae are used:
MeCN Acetonitrile
Cs₂CO₃ Cesium carbonate
DCM Dichloromethane
Et₂O Diethyl ether
DMF N,N-dimethylformamide
DMSO Dimethyl sulfoxide
EtOAc Ethyl acetate
H₂ Hydrogen
HCl Hydrochloric acid
HPLC High performance liquid chromatography
LiOH Lithium hydroxide
MeOH Methanol
MgSO₄ Magnesium sulfate
N₂ Nitrogen
Pd/C Palladium on carbon
Pd(OH)₂ Palladium hydroxide
K₂CO₃ Potassium carbonate
K₃PO₄ Potassium phosphate
NaHCO₃Sodium bicarbonate
NaH Sodium hydride
NaOH Sodium hydroxide
Na₂SO₄ Sodium sulfate
SNAr Aromatic Nucleophilic Substitution
TFA Trifluoroacetic acid
THF Tetrahydrofuran
Et₃N Triethylamine
H₂O Water
RT Room temperature

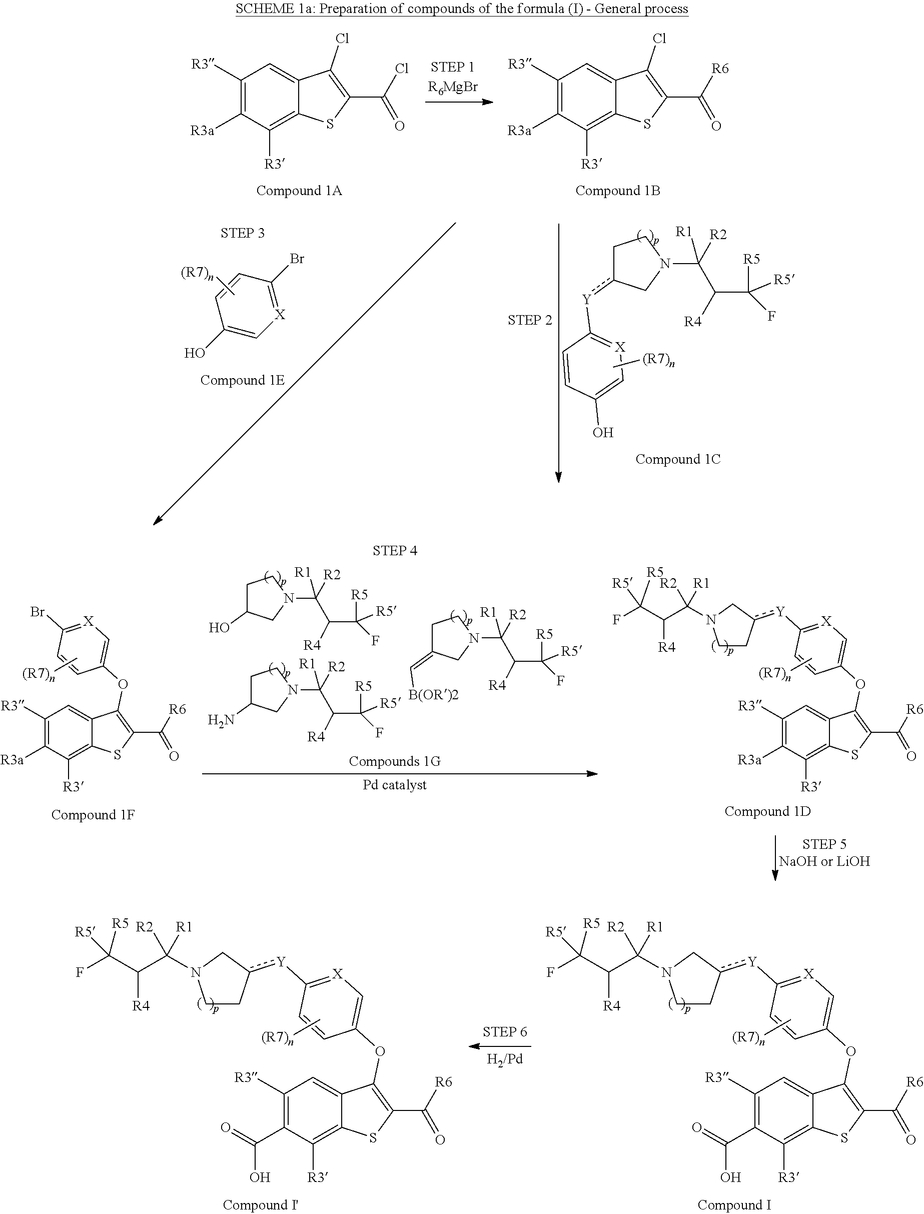

According to SCHEME 1a, in which R3a is a carboxylic ester such as COOMe, COOEt, R1, R2, R3′, R3″, R4, R5, R5′, R6, R7,

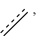

, n, p, X and Y are defined as described above, compound 1A (prepared according to US20130231484), can be converted in STEP 1 to compound 1B by treatment with R6MgBr in solution in THF.

Compound 1B can be converted in STEP 2 to compound 1D by treatment with compound 1C in a SNAr reaction in a solvent such as DMF, in the presence of a base, for example cesium carbonate (Cs₂CO₃), by heating up to reflux of solvent.

Alternatively, compound 1D can be obtained by treatment of compound 1B with compound 1E in STEP 3 in a SNAr reaction in a solvent such as DMF, in the presence of a base, for example sodium hydride (NaH), followed by a coupling reaction in STEP 4 between obtained compound 1F and one of compounds 1G in coupling reaction conditions.

Compound 1D can be converted in STEP 5 to compound I by treatment with an aqueous solution of sodium hydroxide (NaOH) or lithium hydroxide (LiOH) in MeOH or THF. Extraction of the product could give the sodium or lithium salt of compound I. The acidification with an aqueous solution of HCl to pH 6-7 could give the neutral form of compound I. The acidification with an aqueous solution of HCl to pH 1-2 could give the hydrochloride salt of compound I. The purification using HPLC in presence of formic acid or trifluoroacetic acid in the eluent could give the formate or trifluoroacetate salt of compound I.

When Y=—CH═, compound I may be reduced by hydrogenation in STEP 6 with a catalyst, such as Pd/C or Pd(OH)₂/C under hydrogen (H₂) pressure to give the corresponding saturated compound I′.

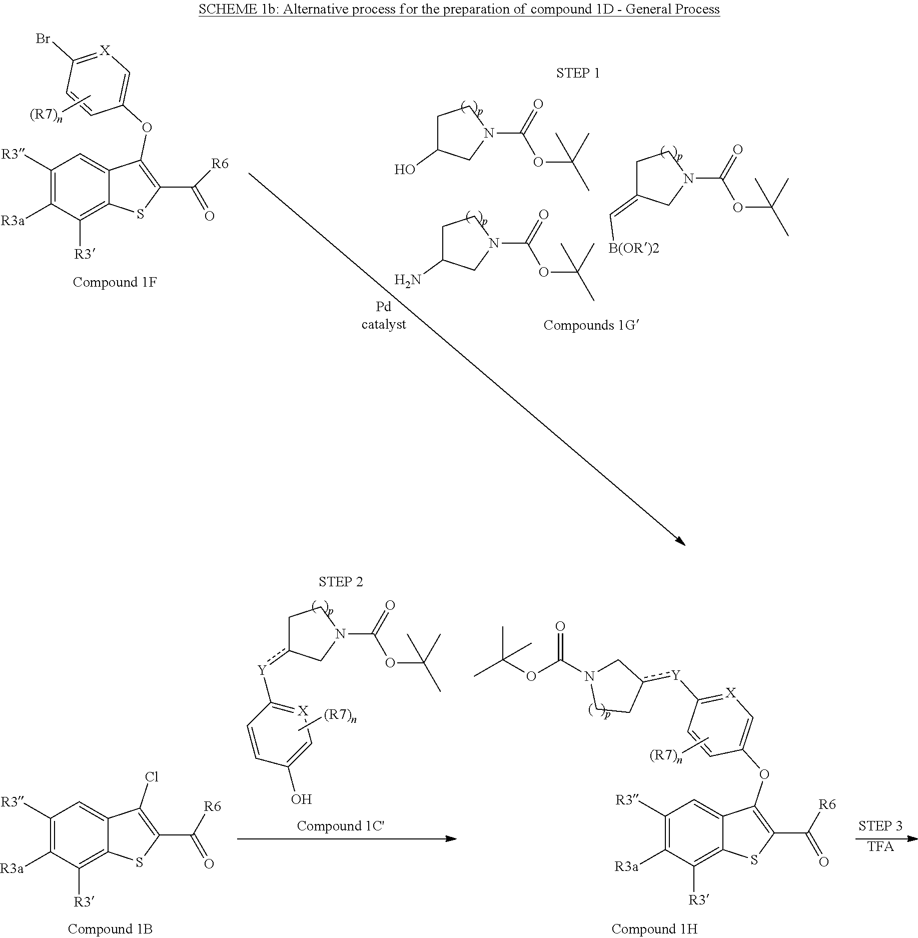

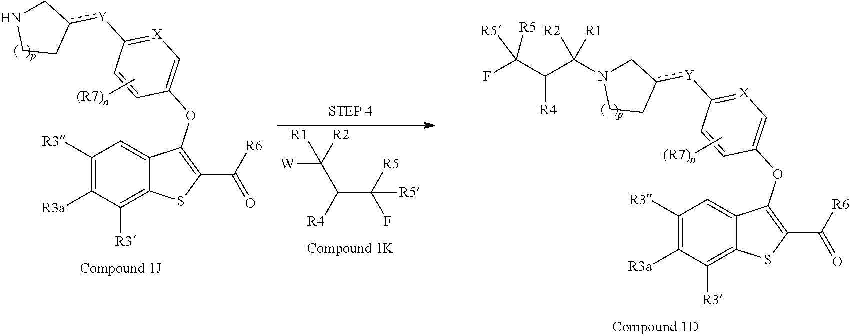

According to SCHEME 1b, in which R3a is carboxylic ester such as COOMe, COOEt, R1, R2, R3′, R3″, R4, R5, R5′, R6, R7,

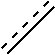

n, p, X and Y are defined as described above, compound 1F can be converted in STEP 1 to compound 1H by coupling reaction with one of compounds 1G′ in coupling reaction conditions.

Alternatively, compound 1H can be obtained by treatment of compound 1B with compound 1C′ in STEP 2 in a SNAr reaction in a solvent such as DMF, in the presence of a base, for example cesium carbonate (Cs₂CO₃), by heating up to reflux of solvent.

Compound 1H can be converted in STEP 3 to compound 1J by treatment with TFA or HCl.

Compound 1J can be converted to compound 1D by the treatment with compound 1K, wherein W is Cl, Br or I or OsO₂R with R=CH₃, PhMe, CF₃ or CF₂CF₂CF₂CF₃ in presence of a base such as potassium carbonate in DMF as a solvent.

Herein is also provided a process for preparing a compound of formula (I) as defined above, wherein a compound of formula 1D

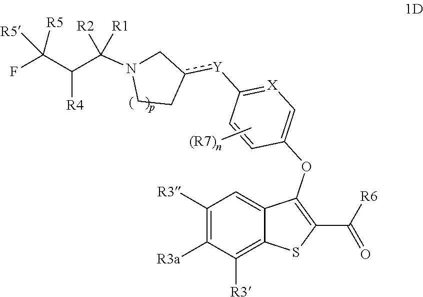

wherein R1, R2, R3′, R3″, R4, R5, R5′, R6, R7,

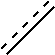

n, p, X and Y are defined as described above and R3a is a carboxylic ester such as COOMe or COOEt is converted to compound of formula (I), in presence of a source of hydroxide ions, such as NaOH or LiOH in solution in methanol or THF, said step being optionally preceded by a step of obtaining compound 1D, wherein a compound of formula 1F

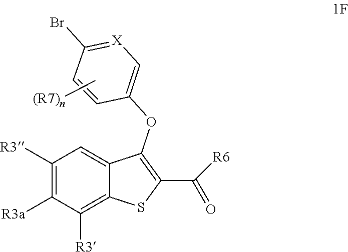

wherein R3′, R3″, R6, R7, n, and X are defined as described above and R3a is a carboxylic ester such as COOMe or COOEt, is subjected to a coupling step with one of compounds 1G
wherein R1, R2, R4, R5, R5′ and p are as defined above.

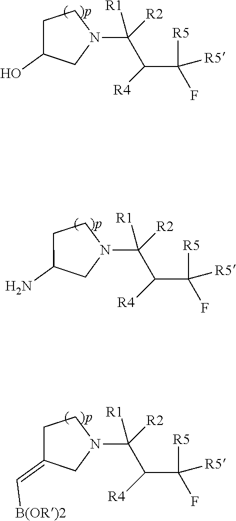

Herein is also provided a process for preparing a compound of formula (I) as defined above, wherein a compound of formula 1D

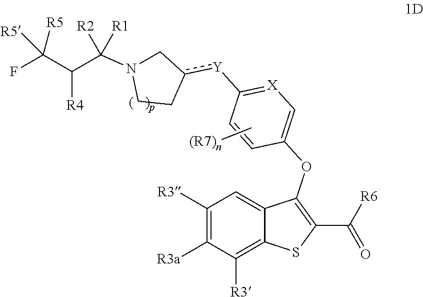

wherein R1, R2, R3′, R3″, R4, R5, R5′, R6, R7,

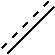

n, p, X and Y are defined as described above and R3a is a carboxylic ester such as COOMe or COOEt is converted to compound of formula (I), in presence of a source of hydroxide ions, such as NaOH or LiOH in solution in methanol or THF, said step being optionally preceded by a step of obtaining compound 1D, wherein a compound of formula 1B

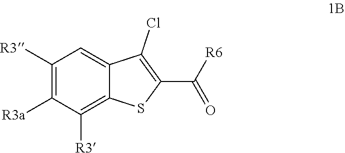

wherein R3′, R3″ and R6 are defined as described above and R3a is a carboxylic ester such as COOMe or COOEt, is subjected to a SNAr reaction in a solvent such as DMF, in the presence of a base, with compound 1C

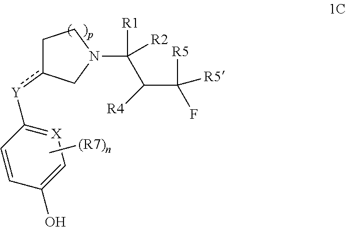

wherein R1, R2, R4, R5, R5′, p, n, R7, X,

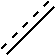

and Y are as defined above.

Herein is also provided a process for preparing a compound of formula (I) as defined above, wherein a compound of formula 1D

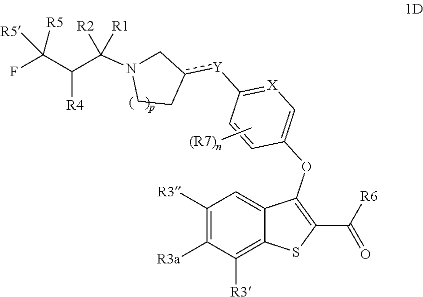

wherein R1, R2, R3′, R3″, R4, R5, R5′, R6, R7, n, p, X,

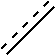

and Y are defined as described above and R3a is a carboxylic ester such as COOMe or COOEt is converted to compound of formula (I), in presence of a source of hydroxide ions, such as NaOH or LiOH in solution in methanol or THF, said step being optionally preceded by a step of obtaining compound 1D, wherein a compound of formula 1J

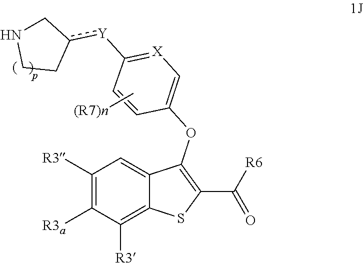

wherein R3′, R3″, Y, p,

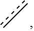

X, R7, n and R6 are defined as described above and R3a is a carboxylic ester such as COOMe or COOEt, is converted in presence of a base with compound 1K

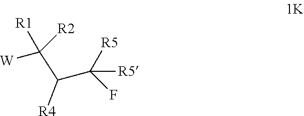

wherein W is Cl, Br or I or OsO₂R with R=CH₃, PhMe, CF₃ or CF₂CF₂CF₂CF₃.

Herein are also provided the intermediate compounds selected from compounds of formula 1D, 1F, 1H, 1J, or any of its pharmaceutically acceptable salt,

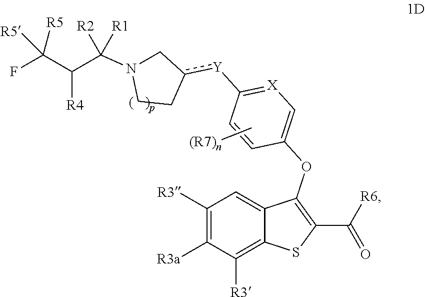

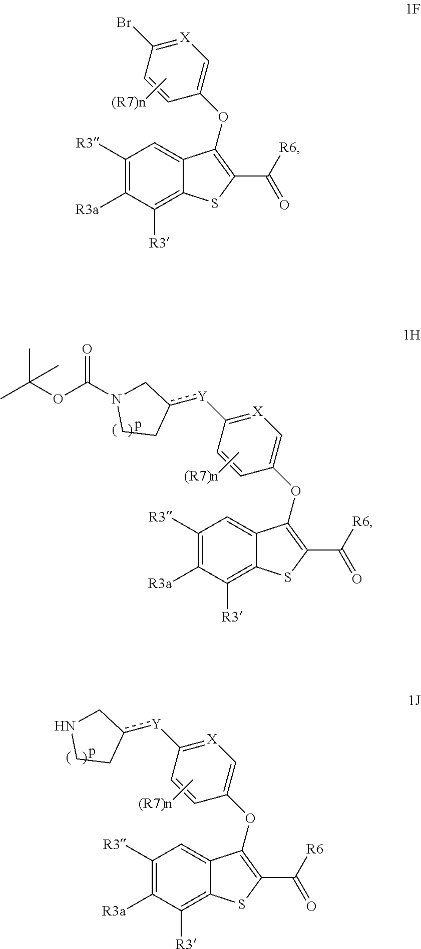

wherein R1, R2, R3′, R3″, R4, R5, R5′, R6, R7, n, p,

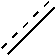

X and Y are defined above and R3a is a carboxylic ester such as COOMe or COOEt.

In another aspect, herein is also provided a process for the preparation of a compound of formula (I), comprising a deprotection step of a compound of formula 1D as defined above, optionally followed by a purification step.

Said purification step may for example consist, as illustrated in step 2 of example 1 herein after, in an acidification step, for example with an aqueous solution of hydrochloric acid.

The 1H NMR Spectra at 400 and 500 MHz were performed on a Bruker Avance DRX-400 and Bruker Avance DPX-500 spectrometer, respectively, with the chemical shifts (8 in ppm) in the solvent dimethyl sulfoxide-d6 (d6-DMSO) referenced at 2.5 ppm at a temperature of 303 K. Coupling constants (J) are given in Hertz.

The liquid chromatography/mass spectra (LC/MS) were obtained on a UPLC Acquity Waters instrument, light scattering detector Sedere and SQD Waters mass spectrometer using UV detection DAD 210-400 nm and flash Acquity UPLC CSH C18 1.7 μm, dimension 2.1×30 mm, mobile phase H₂O+0.1% HCO₂H/CH₃CN+0.1% HCO₂H.

The following tables 1a and 1b comprises respectively specific compounds of formula (I) (name and structure) in accordance with the present disclosure as well their characterization (1H NMR and liquid chromatography/mass).

TABLE 1a

| Ex | Structure | Name |
|---|---|---|
| 1 | 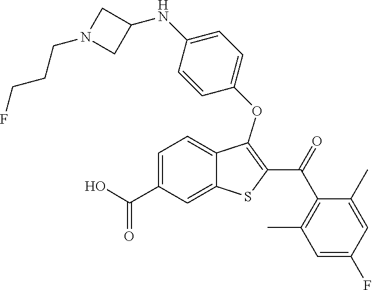 [SMILES: Cc1cc(F)cc(C)c1C(=O)c1sc2cc(C(=O)O)ccc2c1Oc1ccc(NC2CN(CCCF)C2)cc1] | 2-(4-fluoro-2,6- dimethylbenzoyl)-3-(4-((1- (3-fluoropropyl)azetidin-3- yl)amino)phenoxy)benzo[b] thiophene-6-carboxylic acid |
| 2 | 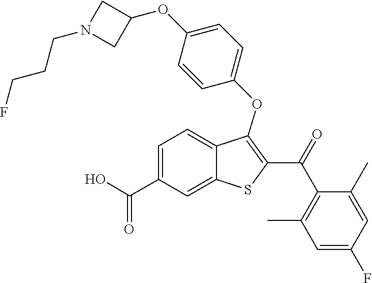 [SMILES: Cc1cc(F)cc(C)c1C(=O)c1sc2cc(C(=O)O)ccc2c1Oc1ccc(OC2CN(CCCF)C2)cc1] | 2-(4-fluoro-2,6- dimethylbenzoyl)-3-(4-((1- (3-fluoropropyl)azetidin-3- yl)oxy)phenoxy)benzo[b] thiophene-6-carboxylic acid |
| 3 | 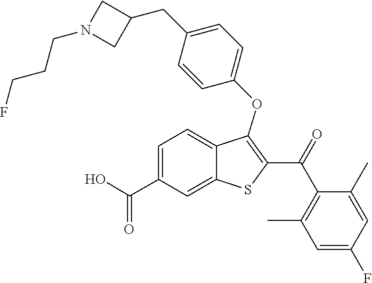 [SMILES: Cc1cc(F)cc(C)c1C(=O)c1sc2cc(C(=O)O)ccc2c1Oc1ccc(CC2CN(CCCF)C2)cc1] | 2-(4-fluoro-2,6- dimethylbenzoyl)-3-(4-((1- (3-fluoropropyl)azetidin-3- yl)methyl)phenoxy)benzo[b] thiophene-6-carboxylic acid |
| 4 | 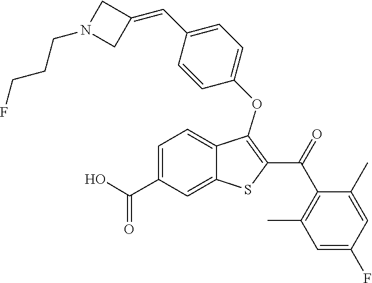 [SMILES: Cc1cc(F)cc(C)c1C(=O)c1sc2cc(C(=O)O)ccc2c1Oc1ccc(C=C2CN(CCCF)C2)cc1] | 2-(4-fluoro-2,6- dimethylbenzoyl)-3-(4-((1- (3-fluoropropyl)azetidin-3- ylidene)methyl)phenoxy) benzo[b]thiophene-6- carboxylic acid |
| 5 | 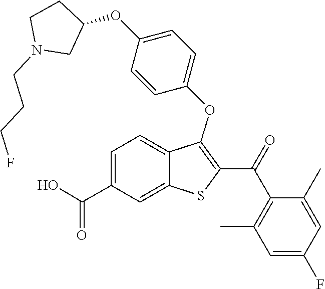 [SMILES: Cc1cc(F)cc(C)c1C(=O)c1sc2cc(C(=O)O)ccc2c1Oc1ccc(O[C@H]2CCN(CCCF)C2)cc1] | (S)-2-(4-fluoro-2,6- dimethylbenzoyl)-3-(4-((1- (3-fluoropropyl)pyrrolidin-3- yl)oxy)phenoxy)benzo[b] thiophene-6-carboxylic acid |
| 6 | 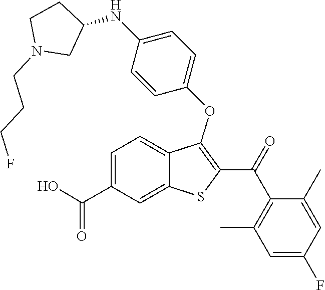 [SMILES: Cc1cc(F)cc(C)c1C(=O)c1sc2cc(C(=O)O)ccc2c1Oc1ccc(N[C@H]2CCN(CCCF)C2)cc1] | (S)-2-(4-fluoro-2,6- dimethylbenzoyl)-3-(4-((1- (3-fluoropropyl)pyrrolidin-3- yl)amino)phenoxy)benzo[b] thiophene-6-carboxylic acid |

(the first column “Ex” corresponds to the compound and example number)

TABLE 1b

| | | | MASS: |
|---|---|---|---|
| | | | LC/MS |
| | Preparation | | (m/z, |
| Ex | Method | NMR | MH+): |
| 1 | A | 1H-NMR (500 MHz, DMSO-d6) δ (ppm): 8.87- | 551 |
| | | 8.47 (m, 1H), 7.86 (dd, J = 8.5, 1.4 Hz, 1H), 7.41 | |
| | | (d, J = 8.5 Hz, 1H), 6.82 (d, J = 9.8 Hz, 2H), 6.37 | |
| | | (d, J = 9.3 Hz, 2H), 6.33 (d, J = 9.1 Hz, 2H), 5.98 | |
| | | (s, 1H), 4.47 (dt, J = 47.3, 5.9 Hz, 2H), 3.92 (s, | |
| | | 1H), 3.78 (t, J = 7.2 Hz, 2H), 2.96 (s, 2H), 2.70- | |
| | | 2.61 (m, 2H), 2.07 (s, 6H), 1.71 (m, 2H) | |
| 2 | −B | 1H NMR (500 MHz, DMSO-d6) δ (ppm): 13.33 | 552 |
| | | (s, 1H), 8.73 (s, 1H), 7.89 (d, J = 9.9 Hz, 1H), 7.50 | |
| | | (d, J = 8.5 Hz, 1H), 6.79 (d, J = 9.8 Hz, 2H), 6.63 | |
| | | (d, J = 9.1 Hz, 2H), 6.45 (d, J = 9.1 Hz, 2H), 4.66 | |
| | | (p, J = 5.7 Hz, 1H), 4.45 (dt, J = 47.4, 6.0 Hz, 2H), | |
| | | 3.72-3.62 (m, 2H), 2.99-2.84 (m, 2H), 2.04 (s, | |
| | | 6H), 1.78-1.52 (m, 2H) | |
| 3 | B | 1H NMR (500 MHz, DMSO-d6) δ (ppm): 8.62 (s, | 550 |
| | | 1H), 7.83 (d, J = 9.9 Hz, 1H), 7.40 (d, J = 8.4 Hz, | |
| | | 1H), 6.88 (d, J = 8.7 Hz, 2H), 6.66 (d, J = 9.9 Hz, | |
| | | 2H), 6.34 (d, J = 8.8 Hz, 2H), 4.39 (dt, J = 47.3, | |
| | | 6.0 Hz, 2H), 3.42-3.36 (m, 2H), 3.27 (s, 5H), 2.96 | |
| | | (s, 2H), 2.66 (d, J = 7.7 Hz, 2H), 2.59 (d, J = 12.8 | |
| | | Hz, 3H), 2.27 (s, 1H), 1.95 (s, 6H), 1.65-1.76 (m, | |
| | | 2H) | |
| 4 | C | 1H NMR (500 MHz, DMSO) δ (ppm): 8.70 (s, | 548 |
| | | 1H), 7.90 (dd, J = 8.5, 1.4 Hz, 1H), 7.49 (d, J = | |
| | | 8.5 Hz, 1H), 6.93 (d, J = 8.9 Hz, 1H), 6.77 (d, J = | |
| | | 9.8 Hz, 1H), 6.50 (d, J = 8.9 Hz, 1H), 6.12 (s, 1H), | |
| | | 4.48 (dt, J = 47.3, 6.0 Hz, 1H), 4.06 (s, 1H), 3.92 | |
| | | (s, 1H), 2.63 (s, 2H), 2.04 (s, 6H), 1.76-1.64 (m, | |
| | | 2H). | |
| 5 | B | 1H NMR (500 MHz, DMSO-d6) δ (ppm): 8.78 | 566 |
| | | (s, 1H), 7.92 (d, J = 8.4 Hz, 1H), 7.53 (d, J = 8.4 | |
| | | Hz, 1H), 6.80 (dd, J = 10.4 Hz, 4H), 6.51 (d, J = | |
| | | 8.6 Hz, 2H), 4.98 (bs, 1H), 4.59 (dt, J = 47.2, 5.7 | |
| | | Hz, 2H), 3.47 (m, 2H), 3.20-3.10 (m, 4H), 2.04 | |
| | | (s, 6H), 2.02-1.95 (m, 2 H), 1.42-1.38 (m, 2H) | |
| 6 | A | 1H-NMR (500 MHz, DMSO-d6) δ (ppm): 8.77 | 565 |
| | | (d, J = 1.4 Hz, 1H), 7.86 (dd, J = 8.5, 1.5 Hz, 1H), | |
| | | 7.42 (d, J = 8.5 Hz, 1H), 6.82 (d, J = 9.8 Hz, 2H), | |
| | | 6.36 (s, 4H), 5.62 (s, 1H), 4.50 (dt, J = 47.4, 6.0 | |
| | | Hz, 2H), 3.82 (bs, 1H), 2.88 (m, 1H), 2.74 (m, | |
| | | 1H), 2.60 (m, 4H), 2.49-2.43 (m, 1H), 2.18 (m, | |
| | | 1H), 1.85 (m, 2H), 1.59 (m, 1H) | |

The examples which follow describe the preparation of some compounds of formula (I) described herein. The numbers of the compounds exemplified below match those given in the Tables 1a and 1b above. All reactions are performed under inert atmosphere, unless otherwise stated.

In the following examples, when the source of the starting products is not specified, it should be understood that said products are known compounds.

### EXAMPLES

### Intermediates

### Intermediate 1: Methyl 3-chloro-2-(4-fluoro-2,6-dimethylbenzoyl)benzo[b]thiophene-6-carboxylate

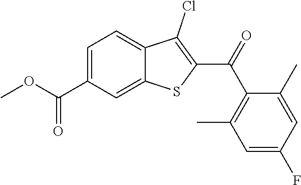 [SMILES: COC(=O)c1ccc2c(Cl)c(C(=O)c3c(C)cc(F)cc3C)sc2c1]

### Step 1:(4-Fluoro-2,6-dimethyl-phenyl)magnesium bromide

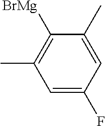

To a suspension of magnesium turnings (0.19 g, 7.83 mmol) in THF (7.61 ml) under nitrogen was dropwise added 2-bromo-5-fluoro-1,3-dimethylbenzene (1.50 g, 7.39 mmol). The reaction mixture was stirred at RT for 4 h. The reaction mixture was directly engaged in the next step without further treatment (considered quantitative).

### Step 2: Methyl 3-chloro-2-(4-fluoro-2,6-dimethylbenzoyl)benzo[b]thiophene-6-carboxylate

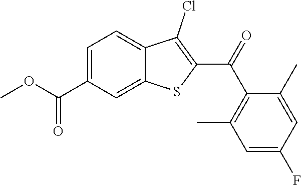 [SMILES: COC(=O)c1ccc2c(Cl)c(C(=O)c3c(C)cc(F)cc3C)sc2c1]

To a solution of methyl 3-chloro-2-(chlorocarbonyl)benzo[b]thiophene-6-carboxylate (prepared according to US20130231484) (2.5 g, 8.65 mmol) in THF (12.5 ml) at 0° C. was dropwise added a solution of (4-fluoro-2,6-dimethyl-phenyl)magnesium bromide (11.3 ml, 11.3 mmol). The reaction mixture was stirred at RT for 12 h. The reaction mixture was acidified with an aqueous 1M solution of HCl (10 ml) at 0° C. (pH was adjusted from 2 to 2.5) and extracted with EtOAc (30 ml), dried over Na₂SO₄, filtered, and concentrated under reduced pressure. The crude was purified by flash chromatography eluting with a gradient of cyclohexane/EtOAc from 100/00 to 85/15 to give 1.95 g (60%) of methyl 3-chloro-2-(4-fluoro-2,6-dimethylbenzoyl)benzo[b]thiophene-6-carboxylate. LC/MS (m/z, MH+): 377

### Intermediate 2: Methyl 3-(4-bromophenoxy)-2-(4-fluoro-2,6-dimethylbenzoyl)benzo[b]thiophene-6-carboxylate

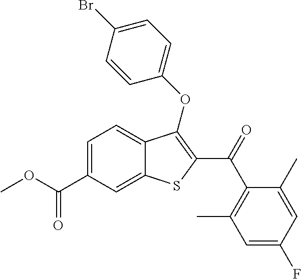 [SMILES: COC(=O)c1ccc2c(Oc3ccc(Br)cc3)c(C(=O)c3c(C)cc(F)cc3C)sc2c1]

To a solution of methyl 3-chloro-2-(4-fluoro-2,6-dimethylbenzoyl)benzo[b]thiophene-6-carboxylate (Intermediate 1) (1.3 g, 3.45 mmol) and 4-bromophenol (1.79 g, 10.35 mmol) in DMF (65 ml) at RT was added NaH 60% in mineral oil (0.41 g, 10.35 mmol). The reaction mixture was stirred at 50° C. for 4 h. The reaction mixture was cooled to RT then an aqueous solution of NaOH (1N) (10 ml) was added. The mixture was extracted with EtOAc (3×30 ml). The organic layers were washed with water (30 ml), dried over anhydrous Na₂SO₄, filtered and concentrated under reduced pressure and the residue obtained was purified by flash chromatography eluting with a gradient of cyclohexane/EtOAc from 100/00 to 90/10 to give 945 mg (53%) of methyl 3-(4-bromophenoxy)-2-(4-fluoro-2,6-dimethylbenzoyl)benzo[b]thiophene-6-carboxylate as a yellow solid.

LC/MS (m/z, MH+): 514

### Intermediate 3: (S)-4-((1-(3-Fluoropropyl)pyrrolidin-3-yl)oxy)phenol

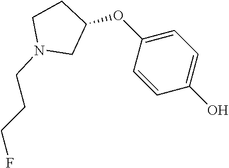 [SMILES: Oc1ccc(O[C@H]2CCN(CCCF)C2)cc1]

### Step 1: (R)-1-(3-Fluoropropyl)pyrrolidin-3-ol

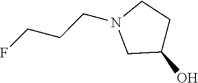

A suspension of (3R)-3-pyrrolidinol (2.00 g, 1.91 mL, 21.8 mmol), 1-fluoro-3-iodopropane (4.28 g, 22.8 mmol) and K₂CO₃ (8.68 g, 62.2 mmol) in MeCN (20 ml) was stirred at 50° C. for 18 hours. The reaction mixture was cooled down to RT and the suspension was filtered through celite. The filtrate was evaporated under reduced pressure and solubilized in DCM (5 ml), then brine (5 ml) was added. The layers were separated. The aqueous layer was extracted further with DCM (3×10 ml). The aqueous phase was concentrated under reduced pressure, solubilized with a saturated aqueous solution of NaHCO₃(pH=9-10) and extracted with a solution of 10% MeOH in DCM (3×10 ml). All the organic layers were combined, dried over Na₂SO₄, filtered, and evaporated under reduced pressure to give 2.11 g (66%) of (S)-1-(3-fluoropropyl)pyrrolidin-3-ol as an orange oil.

LC/MS (m/z, MH+): 148

### Step 2: (S)-3-(4-(Benzyloxy)phenoxy)-1-(3-fluoropropyl)pyrrolidine

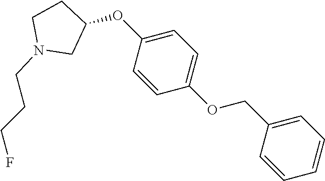 [SMILES: FCCCN1CC[C@H](Oc2ccc(OCc3ccccc3)cc2)C1]

To a solution of 4-(benzyloxy)phenol (1.56 g, 7.8 mmol) in dry THF (11.50 ml) were added (S)-1-(3-fluoropropyl)pyrrolidin-3-ol (1.29 g, 7.8 mmol), triphenylphosphine (3 g, 11.7 mmol) and di-tert-butyl azodicarboxylate (2.70 g, 11.7 mmol). The mixture was stirred under microwave irradiation at 80° C. for 1 h. The reaction mixture was cooled down to RT and concentrated under reduced pressure. The crude was acidified with an aqueous solution of HCl (1 M) (until pH=2-3) and extracted with DCM (3×10 ml). The aqueous phase was basified with an aqueous solution of NaOH (3 M) (until pH=14) and extracted with DCM (3×10 ml). The collected organic layers were combined and washed with brine (5 ml), dried over Na₂SO₄, filtered, and concentrated under reduced pressure. The residue obtained was purified by flash chromatography eluting with a gradient of DCM/isopropanol from 99/01 to 80/20 to give 1.03 g (40%) of (S)-3-(4-(benzyloxy)phenoxy)-1-(3-fluoropropyl)pyrrolidine as a brown sticky oil.

LC/MS (m/z, MH+): 330

### Step 3: (S)-4-((1-(3-Fluoropropyl)pyrrolidin-3-yl)oxy)phenol

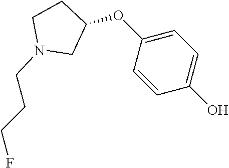 [SMILES: Oc1ccc(O[C@H]2CCN(CCCF)C2)cc1]

To a solution of (S)-3-(4-(benzyloxy)phenoxy)-1-(3-fluoropropyl)pyrrolidine (400 mg, 1.21 mmol) in EtOAc (1.5 ml) was added Pd/C (10% on carbon) (160 mg, 0.15 mmol) at RT. The resulting mixture was purged three times with N₂, then charged with H₂ (1 bar) and stirred at 25° C. for 24 hours. The reaction mixture was filtered through celite and concentrated to dryness to afford 288 mg (99%) of (S)-4-((1-(3-fluoropropyl)pyrrolidin-3-yl)oxy)phenol as a colorless oil.

LC/MS (m/z, MH+): 240

### Intermediate 4: 4-((1-(3-Fluoropropyl)azetidin-3-yl)oxy)phenol

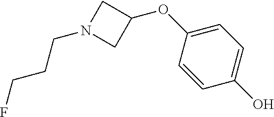 [SMILES: Oc1ccc(OC2CN(CCCF)C2)cc1]

### Step 1: Tert-butyl 3-(4-(benzyloxy)phenoxy)azetidine-1-carboxylate

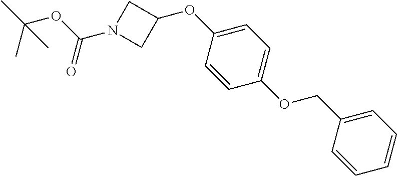 [SMILES: CC(C)(C)OC(=O)N1CC(Oc2ccc(OCc3ccccc3)cc2)C1]

To a solution of 1-Boc-3-iodoazetidine (5.0 g, 17.6 mmol) and 4-(benzyloxy)phenol (2.95 g, 15.0 mmol) in DMF (50 ml) was added Cs₂CO₃ (14.4 g, 44.2 mmol). The reaction mixture was stirred at 140° C. for 3 h. After cooling to RT and addition of water (100 ml) and EtOAc (100 ml), the organic phase was separated and washed with water (40 ml), brine (40 ml), dried over Na₂SO₄ and concentrated under reduced pressure. The residue obtained was purified by flash chromatography eluting with a gradient of cyclo/EtOAc from 95/05 to 85/15, to give 4.1 g (78%) of tert-butyl 3-(4-(benzyloxy)phenoxy)azetidine-1-carboxylate as a beige solid.

LC/MS (m/z, MH+): 356

### Step 2: 3-(4-(Benzyloxy)phenoxy)azetidine

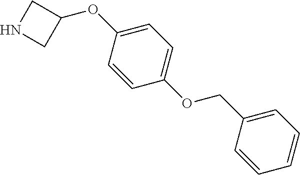 [SMILES: c1ccc(COc2ccc(OC3CNC3)cc2)cc1]

To a solution of tert-butyl 3-(4-(benzyloxy)phenoxy)azetidine-1-carboxylate (3.1 g, 8.72 mmol) in DCM (6.5 ml) at 0° C. was slowly added TFA (6.50 ml, 87.0 mmol). The reaction mixture was allowed to warm to RT and stirred for 4 h. The solution was concentrated under reduced pressure, diluted with DCM (10 ml), and a saturated aqueous solution of NaHCO₃ (20 ml) was added. The aqueous phase was separated and extracted with DCM (3×10 ml). The combined organic layers were washed with brine, dried over Na₂SO₄, filtered, and concentrated under reduced pressure to give 2.22 g (100%) of 3-(4-(benzyloxy)phenoxy)azetidine as a beige powder.

LC/MS (m/z, MH+): 256

### Step 3: 3-(4-(Benzyloxy)phenoxy)-1-(3-fluoropropyl)azetidine

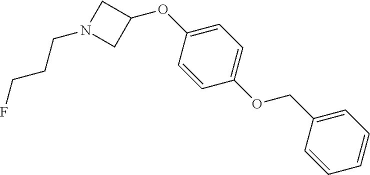 [SMILES: FCCCN1CC(Oc2ccc(OCc3ccccc3)cc2)C1]

A mixture of 3-(4-(benzyloxy)phenoxy)azetidine (2.21 g, 8.70 mmol), 1-fluoro-3-iodopropane (1.79 g, 9.52 mmol) and K₂CO₃ (3.50 g, 24.7 mmol) in MeCN (18 ml) and DMF (8 ml) was stirred at 80° C. for 20 h. After cooling down to RT, the suspension was filtered through a pad of celite. The filtrate was evaporated under reduced pressure and solubilized in EtOAc (30 ml), then brine (20 ml) was added. After decantation, the aqueous layer was extracted with EtOAc (3×10 ml). The combined organic layers were dried over Na₂SO₄, filtered, and evaporated under reduced pressure. The residue obtained was purified by flash chromatography eluting with a gradient of DCM/isopropanol from 98/02 to 80/20 to give 416 mg (16%) of 3-(4-(benzyloxy)phenoxy)-1-(3-fluoropropyl)azetidine as a beige solid.

LC/MS (m/z, MH+): 316

### Step 4: 4-((1-(3-Fluoropropyl)azetidin-3-yl)oxy)phenol

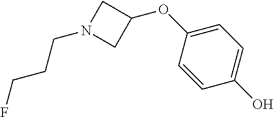 [SMILES: Oc1ccc(OC2CN(CCCF)C2)cc1]

To a solution of 3-(4-(benzyloxy)phenoxy)-1-(3-fluoropropyl)azetidine (270 mg, 0.86 mmol) in MeOH (5 ml) at RT was added Pd(OH)₂ (540 mg, 0.77 mmol). The resulting mixture was purged three time with N₂, then charged with H₂ (1 bar) and stirred at 25° C. for 3 h. The reaction mixture was filtered through celite, and the filtrate was concentrated to dryness to afford 168 mg (87%) of 4-((1-(3-fluoropropyl)azetidin-3-yl)oxy)phenol as a colorless oil.

LC/MS (m/z, MH+): 226

### Intermediate 5: 4-((1-(3-Fluoropropyl)azetidin-3-yl)oxy)phenol

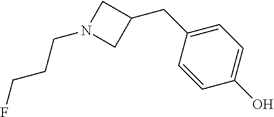 [SMILES: Oc1ccc(CC2CN(CCCF)C2)cc1]

### Step 1: Tert-butyl (E)-3-((2-((4-methoxyphenyl)sulfonyl)hydrazineylidene)methyl)azetidine-1-carboxylate

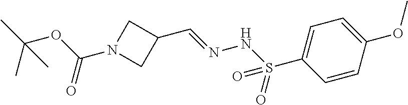 [SMILES: COc1ccc(S(=O)(=O)N/N=C/C2CN(C(=O)OC(C)(C)C)C2)cc1]

To a solution of 4-methoxybenzenesulfonyl hydrazide (8.68 g, 43.0 mmol) in MeOH (86 ml) was added tert-butyl 3-formylazetidine-1-carboxylate (8.75 g, 47.5 mmol). The mixture was stirred at 25° C. until complete conversion. The precipitated white solid was filtered, washed with MeOH (3×7 ml), n-pentane (3×7 ml) and Et₂O (3×7 ml), then dried to give 8.89 g (51%) of tert-butyl (E)-3-((2-((4-methoxyphenyl) sulfonyl) hydrazineylidene)methyl)azetidine-1-carboxylate as a white solid.

LC/MS (m/z, MH+): 370

### Step 2: Tert-butyl 3-(4-(benzyloxy)benzyl)azetidine-1-carboxylate

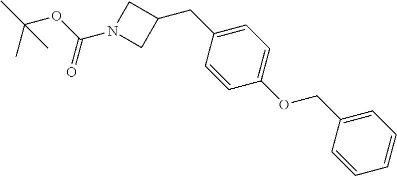 [SMILES: CC(C)(C)OC(=O)N1CC(Cc2ccc(OCc3ccccc3)cc2)C1]

To a solution of tert-butyl (E)-3-((2-((4-methoxyphenyl) sulfonyl) hydrazineylidene)methyl)azetidine-1-carboxylate (7.1 g, 19.1 mmol) in 1,4-dioxane (118 ml) was added 4-benzyloxybenzeneboronic acid (6.50 g, 28.6 mmol) and K₂CO₃ (4.00 g, 28.6 mmol). The mixture was stirred at 110° C. for 24 h. The solution was cooled down to RT. The mixture was diluted with EtOAc (50 ml) and with a saturated aqueous solution of NaHCO₃. The solution was filtered, extracted with EtOAc (5×50 ml) and washed with brine (70 ml). The combined organic layers were dried with Na₂SO₄, filtered, and concentrated under reduced pressure. The residue obtained was purified by flash chromatography eluting with a gradient of cyclohexane/EtOAc from 98/02 to 80/20 to give 0.98 g (15%) of tert-butyl 3-(4-(benzyloxy)benzyl)azetidine-1-carboxylate as a white solid.

LC/MS (m/z, MH+): 354

### Step 3: 3-(4-(Benzyloxy)benzyl)azetidine

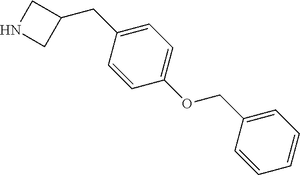 [SMILES: c1ccc(COc2ccc(CC3CNC3)cc2)cc1]

To a solution of tert-butyl 3-(4-(benzyloxy)benzyl)azetidine-1-carboxylate (472 mg, 1.12 mmol) in DCM (1 ml) at 0° C. was slowly added TFA (0.8 ml, 11.2 mmol). The reaction mixture was allowed to warm to RT and stirred for 2 h. The solution was concentrated under reduced pressure, diluted with DCM (2 ml), and a saturated aqueous solution of NaHCO₃ (7 ml) was added. The aqueous layer was separated and extracted with DCM (3×5 ml). The combined organic layers were washed with brine, dried over Na₂SO₄, filtered, and concentrated under reduced pressure to give 192 mg (68%) of 3-(4-(benzyloxy)benzyl)azetidine as a brown sticky oil.

LC/MS (m/z, MH+): 254

### Step 4: 3-(4-(Benzyloxy)benzyl)-1-(3-fluoropropyl)azetidine

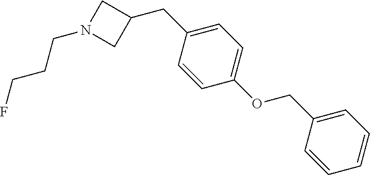 [SMILES: FCCCN1CC(Cc2ccc(OCc3ccccc3)cc2)C1]

To a suspension of 3-(4-(benzyloxy)benzyl)azetidine (227 mg, 0.68 mmol) in DMF (2 ml) were added 1-fluoro-3-iodopropane (153 mg, 0.82 mmol) and K₂CO₃ (264 mg, 1.91 mmol). The reaction mixture was stirred at 50° C. for 2 h. After cooling down to RT, the suspension was filtered through a pad of celite. The filtrate was evaporated under reduced pressure and solubilized in DCM (2 ml), then, brine (2 ml) was added. The layers were separated. The aqueous layer was extracted with DCM (3×5 ml). The combined organic layers were dried over Na₂SO₄, filtered, and evaporated under reduced pressure. The crude was purified by flash chromatography eluting with a gradient of DCM/isopropanol from 98/02 to 80/20 to give 107 mg (50%) of 3-(4-(benzyloxy)benzyl)-1-(3-fluoropropyl)azetidine as a yellow sticky oil.

LC/MS (m/z, MH+): 314

### Step 5: 4-((1-(3-Fluoropropyl)azetidin-3-yl)methyl)phenol

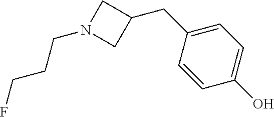 [SMILES: Oc1ccc(CC2CN(CCCF)C2)cc1]

To a N₂-purged suspension of 3-(4-(benzyloxy)benzyl)-1-(3-fluoropropyl)azetidine (132 mg, 0.34 mmol) in MeOH (3.5 ml) was added Pd(OH)₂ (66 mg, 0.10 mmol) at 25° C. The resulting mixture was purged three times with N₂, then charged with H₂ (1 bar) and stirred at 25° C. for 24 h. The reaction mixture was filtered through celite and concentrated to dryness to afford 76 mg (100%) of 4-((1-(3-fluoropropyl)azetidin-3-yl)methyl)phenol as a colorless oil.

LC/MS (m/z, MH+): 224

### Intermediate 6: 1-(3-Fluoropropyl)azetidin-3-amine

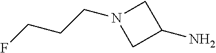

### Step 1: Tert-butyl (1-(3-fluoropropyl)azetidin-3-yl)carbamate

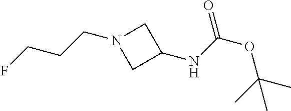 [SMILES: CC(C)(C)OC(=O)NC1CN(CCCF)C1]

A mixture of tert-butyl N-(azetidin-3-yl)carbamate hydrochloride (5 g, 23.48 mmol), 1-fluoro-3-iodopropane (4.86 g, 25.83 mmol) and K₂CO₃ (8.19 g, 58.7 mmol) in THF (50 ml) and water (0.21 g, 0.21 ml, 11.74 mmol). The reaction mixture was stirred at 70° C. for 5 h. The reaction mixture was allowed down to RT. Water (20 ml) and the reaction mixture was extracted with EtOAc (2×20 ml). The organic layers were dried over anhydrous Na₂SO₄ filtered and concentrated under reduced pressure 7 g (78%) of Tert-butyl (1-(3-fluoropropyl)azetidin-3-yl)carbamate.

LC/MS (m/z, MH+): 233

### Step 2:1-(3-Fluoropropyl)azetidin-3-amine

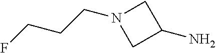

Tert-butyl (1-(3-fluoropropyl)azetidin-3-yl)carbamate (700 mg, 3.013 mmol) was dissolved in DCM (3 ml). Hydrochloric acid solution (2 M) in Et₂O (30 ml, 60.3 mmol) was added and the reaction mixture was stirred at RT for 18 h. The reaction mixture was concentrated under reduced pressure. The crude was triturated with Et₂O (5 ml). The white solid was dissolved in MeOH (20 ml), Amberlyst A26 (960 mg) was added and the mixture was stirred at RT for 12 hours. The resin was filtered, and the filtrate was concentrated under reduced pressure to give 400 mg (100%) of 1-(3-fluoropropyl)azetidin-3-amine as a yellow oil. LC/MS (m/z, MH+): 133

### Intermediate 7: (S)-1-(3-Fluoropropyl)pyrrolidin-3-amine

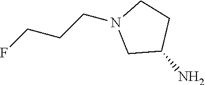

### Step 1: Tert-butyl (S)-(1-(3-fluoropropyl)pyrrolidin-3-yl)carbamate

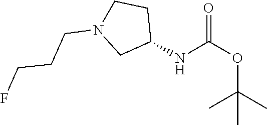 [SMILES: CC(C)(C)OC(=O)N[C@H]1CCN(CCCF)C1]

Step 1 of Intermediate 7 was prepared following a similar procedure to that of step 1 of Intermediate 6 from tert-butyl (S)-pyrrolidin-3-ylcarbamate and 1-fluoro-3-iodopropane to give 6.03 g (91%) of tert-butyl (S)-(1-(3-fluoropropyl)pyrrolidin-3-yl)carbamate.

LC/MS (m/z, MH+): 247

### Step 2: (S)-1-(3-Fluoropropyl)pyrrolidin-3-amine

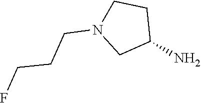

Step 2 of Intermediate 7 was prepared following a similar procedure to that of step 2 of Intermediate 6 from tert-butyl (S)-(1-(3-fluoropropyl)pyrrolidin-3-yl)carbamate to give 237 mg (40%) of (S)-1-(3-fluoropropyl)pyrrolidin-3-amine.

LC/MS (m/z, MH+): 147

### Intermediate 8: 1-(3-Fluoropropyl)-3-((4,4,5,5-tetramethyl-1,3,2-dioxaborolan-2-yl)methylene)azetidine

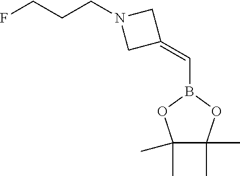 [SMILES: CC1(C)OB(C=C2CN(CCCF)C2)OC1(C)C]

### Step 1: 3-((4,4,5,5-Tetramethyl-1,3,2-dioxaborolan-2-yl)methylene)azetidine, 2,2,2-trifluoroacetic Acid

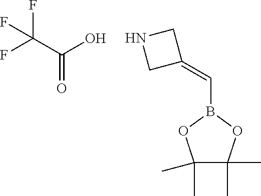

TFA (2.89 mL, 38.96 mmol, 5 eq.) was added at RT to a solution of commercially available tert-butyl 3-((4,4,5,5-tetramethyl-1,3,2-dioxaborolan-2-yl)methylene)azetidine-1-carboxylate (2.3 g, 7.79 mmol) in DCM (20 ml). The solution was stirred for 18 h. The crude oil was evaporated under reduced pressure to give 2.67 g (crude) of 3-((4,4,5,5-tetramethyl-1,3,2-dioxaborolan-2-yl)methylene)azetidine, 2,2,2-trifluoroacetic acid.

LC/MS (m/z, MH+): 196

### Step 2:1-(3-Fluoropropyl)-3-((4,4,5,5-tetramethyl-1,3,2-dioxaborolan-2-yl)methylene)azetidine

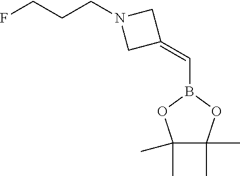 [SMILES: CC1(C)OB(C=C2CN(CCCF)C2)OC1(C)C]

Step 2 of Intermediate 8 was prepared following a similar procedure to that of step 3 of Intermediate 4 from 3-((4,4,5,5-tetramethyl-1,3,2-dioxaborolan-2-yl)methylene)azetidine, 2,2,2-trifluoroacetic acid and 1-fluoro-3-iodopropane to give 1.05 g (60%) of 1-(3-fluoropropyl)-3-((4,4,5,5-tetramethyl-1,3,2-dioxaborolan-2-yl)methylene)azetidine.

LC/MS (m/z, MH+): 256

### Intermediate 9: Methyl 2-(4-fluoro-2,6-dimethylbenzoyl)-3-(4-((1-(3-fluoropropyl)azetidin-3-ylidene)methyl)phenoxy)benzo[b]thiophene-6-carboxylate

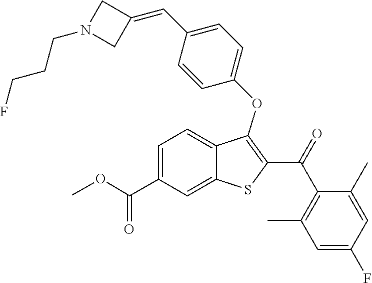 [SMILES: COC(=O)c1ccc2c(Oc3ccc(C=C4CN(CCCF)C4)cc3)c(C(=O)c3c(C)cc(F)cc3C)sc2c1]

### Method 1

### Step 1: Tert-butyl 3-(4-((2-(4-fluoro-2,6-dimethylbenzoyl)-6-(methoxycarbonyl)benzo[b]thiophen-3-yl)oxy)benzylidene)azetidine-1-carboxylate

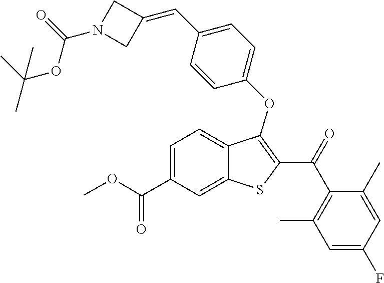 [SMILES: COC(=O)c1ccc2c(Oc3ccc(C=C4CN(C(=O)OC(C)(C)C)C4)cc3)c(C(=O)c3c(C)cc(F)cc3C)sc2c1]

To a solution of methyl 3-(4-bromophenoxy)-2-(4-fluoro-2,6-dimethylbenzoyl)benzo[b]thiophene-6-carboxylate (Intermediate 2) (800 mg, 1.56 mmol), tert-butyl 3-methylideneazetidine-1-carboxylate (791 mg, 4.67 mmol) and Et₃N (0.43 mL, 3.12 mmol) in 1,4-dioxane (12 ml) were added tri-tert-butylphosphonium tetrafluoroborate (135.6 mg, 0.47 mmol) and tris(dibenzylideneacetone) dipalladium (0) (200 mg, 0.22 mmol). The reaction mixture was stirred at 50° C. for 72 h. The reaction mixture was cooled down to RT, then concentrated under reduced pressure. The crude was purified by flash chromatography eluting with a gradient of cyclohexane/DCM from 100:00 to 00:100 to give 480 mg (51%) of tert-butyl 3-(4-((2-(4-fluoro-2,6-dimethylbenzoyl)-6-(methoxycarbonyl)benzo[b]thiophen-3-yl)oxy)benzylidene)azetidine-1-carboxylate as a yellow oil.

LC/MS (m/z, MH+): 602

### Step 2: Methyl 2-(4-fluoro-2,6-dimethylbenzoyl)-3-(4-((1-(3-fluoropropyl)azetidin-3-ylidene)methyl)phenoxy)benzo[b]thiophene-6-carboxylate

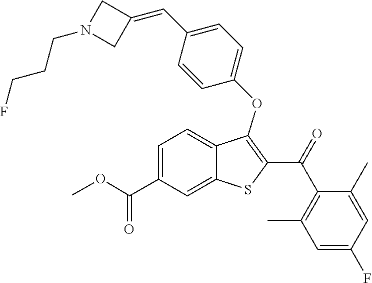 [SMILES: COC(=O)c1ccc2c(Oc3ccc(C=C4CN(CCCF)C4)cc3)c(C(=O)c3c(C)cc(F)cc3C)sc2c1]

To a solution of tert-butyl 3-(4-((2-(4-fluoro-2,6-dimethylbenzoyl)-6-(methoxycarbonyl)benzo[b]thiophen-3-yl)oxy)benzylidene)azetidine-1-carboxylate (330 mg, 0.44 mmol) in DCM (3.5 ml) at RT was added TFA (0.33 ml, 4.39 mmol). The mixture was stirred at RT for 3 h. The reaction was quenched with an aqueous solution of K₂CO₃ (1.21 g, 8.80 mmol) in H₂O (1.50 ml) and 1-fluoro-3-iodopropane (99 mg, 0.53 mmol) was added to the mixture and allowed to stir at RT for 4 h. The reaction was extracted with DCM (3×3 ml), washed with brine (3 ml), dried over Na₂SO₄, filtered and concentrated under reduced pressure. The crude was purified by flash chromatography eluting with DCM/isopropanol from 100/00 to 80/20 to give 17 mg (6%) of methyl 2-(4-fluoro-2,6-dimethylbenzoyl)-3-(4-((1-(3-fluoropropyl)azetidin-3-ylidene)methyl)phenoxy)benzo[b]thiophene-6-carboxylate.

LC/MS (m/z, MH+): 562

### Method 2

### Methyl 2-(4-fluoro-2,6-dimethylbenzoyl)-3-(4-((1-(3-fluoropropyl)azetidin-3-ylidene)methyl)phenoxy)benzo[b]thiophene-6-carboxylate

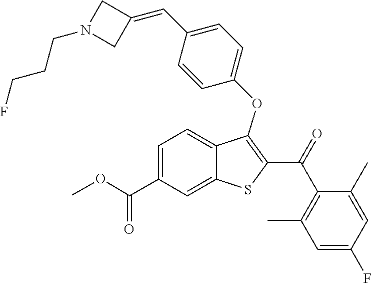 [SMILES: COC(=O)c1ccc2c(Oc3ccc(C=C4CN(CCCF)C4)cc3)c(C(=O)c3c(C)cc(F)cc3C)sc2c1]

A mixture of methyl 3-(4-bromophenoxy)-2-(4-fluoro-2,6-dimethylbenzoyl)benzo[b]thiophene-6-carboxylate (Intermediate 2) (150 mg, 0.29 mmol), K₃PO₄ (186 mg, 0.88 mmol), SPhos (6 mg, 0.015 mmol), palladium acetate (2.42 mg, 0.015 mmol), and 1-(3-fluoropropyl)-3-((4,4,5,5-tetramethyl-1,3,2-dioxaborolan-2-yl)methylene)azetidine (Intermediate 8) (447 mg, 0.88 mmol) in THF (0.48 ml) and H₂O (0.010 ml) was heated at 40° C. for 18 h. After cooling down to RT, MgSO₄ was added. The crude mixture was diluted with EtOAc then filtered off. The filtrate was evaporated under reduced pressure then purified by flash chromatography eluting with DCM/isopropanol from 100/00 to 80/20 to give to give 117 mg (71%) of methyl 2-(4-fluoro-2,6-dimethylbenzoyl)-3-(4-((1-(3-fluoropropyl)azetidin-3-ylidene)methyl)phenoxy)benzo[b]thiophene-6-carboxylate.

LC/MS (m/z, MH+): 562

### EXAMPLES

### Method A:

### Example 1: 2-(4-Fluoro-2,6-dimethylbenzoyl)-3-(4-((1-(3-fluoropropyl)azetidin-3-yl)amino)phenoxy)benzo[b]thiophene-6-carboxylic Acid

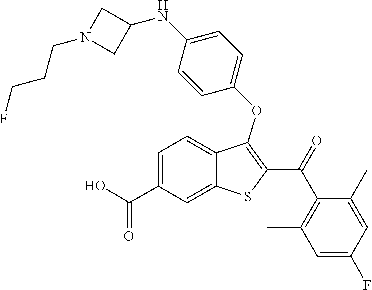 [SMILES: Cc1cc(F)cc(C)c1C(=O)c1sc2cc(C(=O)O)ccc2c1Oc1ccc(NC2CN(CCCF)C2)cc1]

### Step 1: Methyl 2-(4-fluoro-2,6-dimethylbenzoyl)-3-(4-((1-(3-fluoropropyl)azetidin-3-yl)amino)phenoxy)benzo[b]thiophene-6-carboxylate

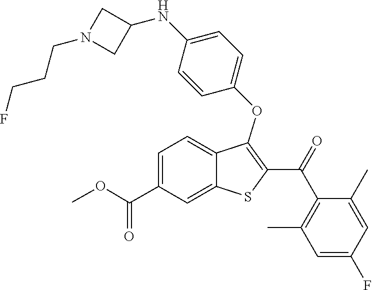 [SMILES: COC(=O)c1ccc2c(Oc3ccc(NC4CN(CCCF)C4)cc3)c(C(=O)c3c(C)cc(F)cc3C)sc2c1]

A mixture of 1-(3-fluoropropyl)azetidin-3-amine (Intermediate 6) (320 mg, 2.42 mmol), methyl 3-(4-bromophenoxy)-2-(4-fluoro-2,6-dimethylbenzoyl)benzo[b]thiophene-6-carboxylate (Intermediate 2) (414 mg, 0.807 mmol), Cs₂CO₃ (661 mg, 2.02 mmol), BrettPhos (87 mg, 0.16 mmol) and BrettPhos Pd G3 (73 mg, 0.081 mmol) in dioxane (7 ml) was stirred at 100° C. for 12 h. After cooling to RT, the reaction mixture was concentrated under reduced pressure and the residue obtained was purified by flash chromatography eluting with a gradient of DCM/isopropanol from 100/00 to 90/10 to give 270 mg (59%) of methyl 2-(4-fluoro-2,6-dimethylbenzoyl)-3-(4-((1-(3-fluoropropyl)azetidin-3-yl)amino)phenoxy)benzo[b]thiophene-6-carboxylate as an orange solid.

LC/MS (m/z, MH+): 565

### Step 2: 2-(4-Fluoro-2,6-dimethylbenzoyl)-3-(4-((1-(3-fluoropropyl)azetidin-3-yl)amino)phenoxy)benzo[b]thiophene-6-carboxylic Acid

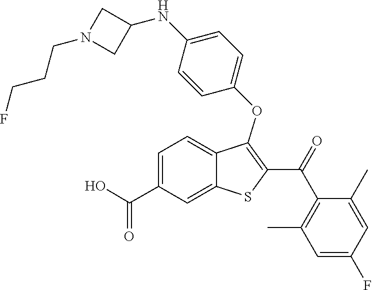 [SMILES: Cc1cc(F)cc(C)c1C(=O)c1sc2cc(C(=O)O)ccc2c1Oc1ccc(NC2CN(CCCF)C2)cc1]

To a solution of methyl 2-(4-fluoro-2,6-dimethylbenzoyl)-3-(4-((1-(3-fluoropropyl)azetidin-3-yl)amino)phenoxy)benzo[b]thiophene-6-carboxylate (230 mg, 0.41 mmol) in mixture of water (2 ml) and THF (2 ml) was added LiOH·H₂O (85 mg, 2.04 mmol). The mixture was stirred at 60° C. for 10 hours. The mixture was acidified with an aqueous solution of HCl (1N) (2 ml) until pH=2. The mixture was extracted with EtOAc (3×10 mL). The organic layers were washed with water (10 ml), dried over anhydrous Na₂SO₄ filtered and concentrated under reduced pressure and the residue obtained was purified by HPLC (C18, Interchim, IR_50SI-F0012, water/MeCN from 98/2 to 0/100) to give 120 mg (54%) of 2-(4-fluoro-2,6-dimethylbenzoyl)-3-(4-((1-(3-fluoropropyl)azetidin-3-yl)amino)phenoxy)benzo[b]thiophene-6-carboxylic acid as a yellow solid.

### Example 6: (S)-2-(4-Fluoro-2,6-dimethylbenzoyl)-3-(4-((1-(3-fluoropropyl)pyrrolidin-3-yl)amino)phenoxy)benzo[b]thiophene-6-carboxylic Acid

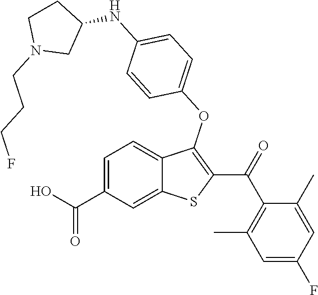 [SMILES: Cc1cc(F)cc(C)c1C(=O)c1sc2cc(C(=O)O)ccc2c1Oc1ccc(N[C@H]2CCN(CCCF)C2)cc1]

### Step 1: Methyl (S)-2-(4-fluoro-2,6-dimethylbenzoyl)-3-(4-((1-(3-fluoropropyl)pyrrolidin-3-yl)amino)phenoxy)benzo[b]thiophene-6-carboxylate

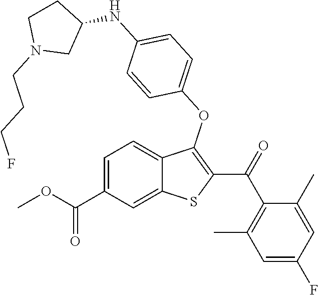 [SMILES: COC(=O)c1ccc2c(Oc3ccc(N[C@H]4CCN(CCCF)C4)cc3)c(C(=O)c3c(C)cc(F)cc3C)sc2c1]

Step 1 of Example 6 was prepared following a similar procedure to that of step 1 of Example 1 from methyl 3-(4-bromophenoxy)-2-(4-fluoro-2,6-dimethylbenzoyl)benzo[b]thiophene-6-carboxylate (Intermediate 2) and (S)-1-(3-fluoropropyl)pyrrolidin-3-amine (Intermediate 7) to give 155 mg (51%) of methyl (S)-2-(4-fluoro-2,6-dimethylbenzoyl)-3-(4-((1-(3-fluoropropyl)pyrrolidin-3-yl)amino)phenoxy)benzo[b]thiophene-6-carboxylate.

LC/MS (m/z, MH+): 579

### Step 2: (S)-2-(4-Fluoro-2,6-dimethylbenzoyl)-3-(4-((1-(3-fluoropropyl)pyrrolidin-3-yl)amino)phenoxy)benzo[b]thiophene-6-carboxylic Acid

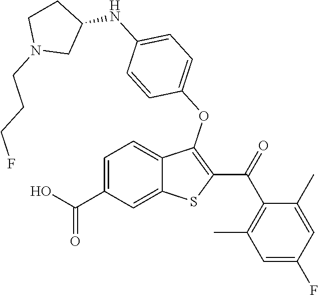 [SMILES: Cc1cc(F)cc(C)c1C(=O)c1sc2cc(C(=O)O)ccc2c1Oc1ccc(N[C@H]2CCN(CCCF)C2)cc1]

Step 2 of Example 6 was prepared following a similar procedure to that of step 2 of Example 1 from methyl (S)-2-(4-fluoro-2,6-dimethylbenzoyl)-3-(4-((1-(3-fluoropropyl)pyrrolidin-3-yl)amino)phenoxy)benzo[b]thiophene-6-carboxylate to give 110 mg (59%) of (S)-2-(4-fluoro-2,6-dimethylbenzoyl)-3-(4-((1-(3-fluoropropyl)pyrrolidin-3-yl)amino)phenoxy)benzo[b]thiophene-6-carboxylic acid.

### Method B:

### Example 2: 2-(4-Fluoro-2,6-dimethylbenzoyl)-3-(4-((1-(3-fluoropropyl)azetidin-3-yl)oxy)phenoxy)benzo[b]thiophene-6-carboxylic Acid

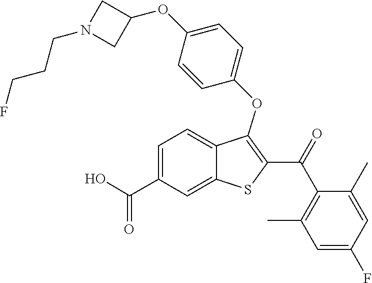 [SMILES: Cc1cc(F)cc(C)c1C(=O)c1sc2cc(C(=O)O)ccc2c1Oc1ccc(OC2CN(CCCF)C2)cc1]

### Step 1: Methyl 2-(4-fluoro-2,6-dimethylbenzoyl)-3-(4-((1-(3-fluoropropyl)azetidin-3-yl)oxy)phenoxy)benzo[b]thiophene-6-carboxylate

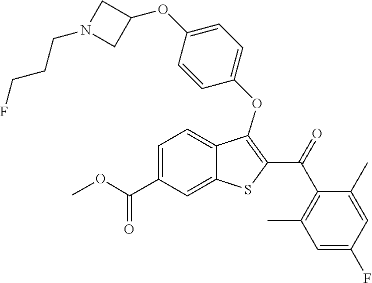 [SMILES: COC(=O)c1ccc2c(Oc3ccc(OC4CN(CCCF)C4)cc3)c(C(=O)c3c(C)cc(F)cc3C)sc2c1]

To a solution of methyl 3-chloro-2-(4-fluoro-2,6-dimethylbenzoyl)benzo[b]thiophene-6-carboxylate (Intermediate 1) (120 mg, 0.32 mmol) in DMF (5.5 ml) were added fluoropropyl)azetidin-3-yl)oxy)phenol (Intermediate 4) (185 mg, 0.79 mmol) and Cs₂CO₃ (251 mg, 0.77 mmol). The reaction was stirred at 100° C. for 2 h. The solution was cooled down to RT, then brine (3 ml) was added. The mixture was concentrated under reduced pressure. The crude was extracted with EtOAc (3×5 ml). The combined organic layers were washed with brine (2 ml), dried over anhydrous Na₂SO₄ filtered and concentrated under reduced pressure. The residue obtained was purified by flash chromatography eluting with a gradient of DCM/isopropanol from 98/02 to 80/20 to give 120 mg (67%) of methyl 2-(4-fluoro-2,6-dimethylbenzoyl)-3-(4-((1-(3-fluoropropyl)azetidin-3-yl)oxy)phenoxy)benzo[b]thiophene-6-carboxylate as a yellow sticky oil.

LC/MS (m/z, MH+): 566

### Step 2: 2-(4-Fluoro-2,6-dimethylbenzoyl)-3-(4-((1-(3-fluoropropyl)azetidin-3-yl)oxy)phenoxy)benzo[b]thiophene-6-carboxylic Acid

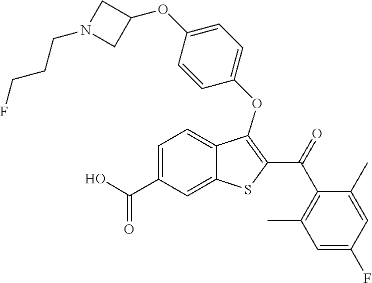 [SMILES: Cc1cc(F)cc(C)c1C(=O)c1sc2cc(C(=O)O)ccc2c1Oc1ccc(OC2CN(CCCF)C2)cc1]

Step 2 of Example 2 was prepared following a similar procedure to that of step 2 of Example 1 from methyl 2-(4-fluoro-2,6-dimethylbenzoyl)-3-(4-((1-(3-fluoropropyl)azetidin-3-yl)oxy)phenoxy)benzo[b]thiophene-6-carboxylate to give 49 mg (56%) of 2-(4-fluoro-2,6-dimethylbenzoyl)-3-(4-((1-(3-fluoropropyl)azetidin-3-yl)oxy)phenoxy)benzo[b]thiophene-6-carboxylic acid.

### Example 3: 2-(4-Fluoro-2,6-dimethylbenzoyl)-3-(4-((1-(3-fluoropropyl)azetidin-3-yl)methyl)phenoxy)benzo[b]thiophene-6-carboxylic Acid

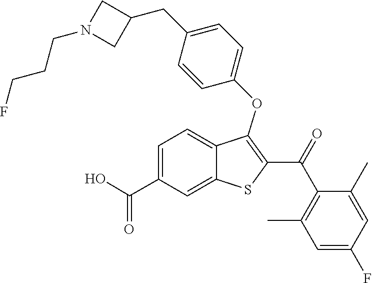 [SMILES: Cc1cc(F)cc(C)c1C(=O)c1sc2cc(C(=O)O)ccc2c1Oc1ccc(CC2CN(CCCF)C2)cc1]

### Step 1: Methyl 2-(4-fluoro-2,6-dimethylbenzoyl)-3-(4-((1-(3-fluoropropyl)azetidin-3-yl)methyl)phenoxy)benzo[b]thiophene-6-carboxylate

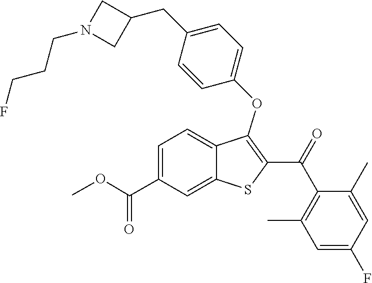 [SMILES: COC(=O)c1ccc2c(Oc3ccc(CC4CN(CCCF)C4)cc3)c(C(=O)c3c(C)cc(F)cc3C)sc2c1]

Step 1 of Example 3 was prepared following a similar procedure to that of step 1 of Example 2 from methyl 3-chloro-2-(4-fluoro-2,6-dimethylbenzoyl)benzo[b]thiophene-6-carboxylate (Intermediate 1) and 4-((1-(3-fluoropropyl)azetidin-3-yl)methyl)phenol (Intermediate 5) to give 58 mg (82%) of methyl 2-(4-fluoro-2,6-dimethylbenzoyl)-3-(4-((1-(3-fluoropropyl)azetidin-3-yl)methyl)phenoxy)benzo[b]thiophene-6-carboxylate.

LC/MS (m/z, MH+): 579

### Step 2: 2-(4-Fluoro-2,6-dimethylbenzoyl)-3-(4-((1-(3-fluoropropyl)azetidin-3-yl)methyl)phenoxy)benzo[b]thiophene-6-carboxylic Acid

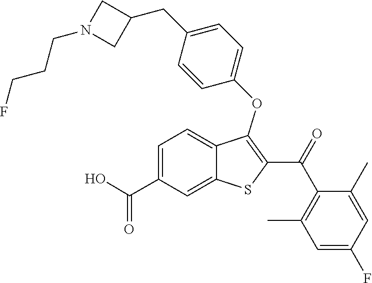 [SMILES: Cc1cc(F)cc(C)c1C(=O)c1sc2cc(C(=O)O)ccc2c1Oc1ccc(CC2CN(CCCF)C2)cc1]

Step 2 of Example 3 was prepared following a similar procedure to that of step 2 of Example 1 from methyl 2-(4-fluoro-2,6-dimethylbenzoyl)-3-(4-((1-(3-fluoropropyl)azetidin-3-yl)methyl)phenoxy)benzo[b]thiophene-6-carboxylate to give 13 mg (23%) of 2-(4-fluoro-2,6-dimethylbenzoyl)-3-(4-((1-(3-fluoropropyl)azetidin-3-yl)methyl)phenoxy)benzo[b]thiophene-6-carboxylic acid.

### Example 5: (S)-2-(4-Fluoro-2,6-dimethylbenzoyl)-3-(4-((1-(3-fluoropropyl)pyrrolidin-3-yl)oxy)phenoxy)benzo[b]thiophene-6-carboxylic Acid

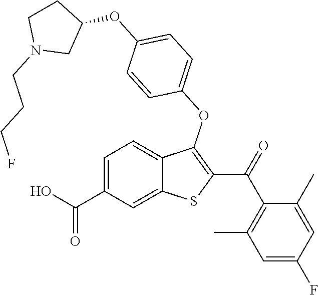 [SMILES: Cc1cc(F)cc(C)c1C(=O)c1sc2cc(C(=O)O)ccc2c1Oc1ccc(O[C@H]2CCN(CCCF)C2)cc1]

### Step 1: Methyl (S)-2-(4-fluoro-2,6-dimethylbenzoyl)-3-(4-((1-(3-fluoropropyl)pyrrolidin-3-yl)oxy)phenoxy)benzo[b]thiophene-6-carboxylate

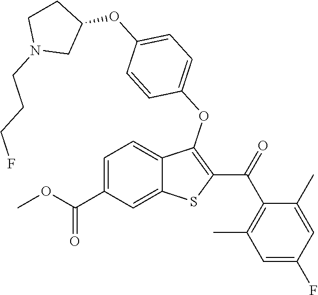 [SMILES: COC(=O)c1ccc2c(Oc3ccc(O[C@H]4CCN(CCCF)C4)cc3)c(C(=O)c3c(C)cc(F)cc3C)sc2c1]

Step 1 of Example 5 was prepared following a similar procedure to that of step 1 of Example 2 from methyl 3-chloro-2-(4-fluoro-2,6-dimethylbenzoyl)benzo[b]thiophene-6-carboxylate (Intermediate 1) and (S)-4-((1-(3-fluoropropyl)pyrrolidin-3-yl)oxy)phenol (Intermediate 3) to give 181 mg (84%) of methyl (S)-2-(4-fluoro-2,6-dimethylbenzoyl)-3-(4-((1-(3-fluoropropyl)pyrrolidin-3-yl)oxy)phenoxy)benzo[b]thiophene-6-carboxylate. LC/MS (m/z, MH+): 579

### Step 2: (S)-2-(4-Fluoro-2,6-dimethylbenzoyl)-3-(4-((1-(3-fluoropropyl)pyrrolidin-3-yl)oxy)phenoxy)benzo[b]thiophene-6-carboxylic Acid

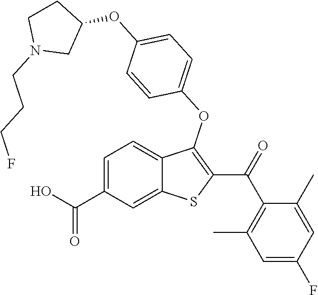 [SMILES: Cc1cc(F)cc(C)c1C(=O)c1sc2cc(C(=O)O)ccc2c1Oc1ccc(O[C@H]2CCN(CCCF)C2)cc1]

Step 2 of Example 5 was prepared following a similar procedure to that of step 2 of Example 1 from methyl (S)-2-(4-fluoro-2,6-dimethylbenzoyl)-3-(4-((1-(3-fluoropropyl)pyrrolidin-3-yl)oxy)phenoxy)benzo[b]thiophene-6-carboxylate to give 55 mg (33%) of (S)-2-(4-fluoro-2,6-dimethylbenzoyl)-3-(4-((1-(3-fluoropropyl)pyrrolidin-3-yl)oxy)phenoxy)benzo[b]thiophene-6-carboxylic acid.

### Method C:

### Example 4: 2-(4-Fluoro-2,6-dimethylbenzoyl)-3-(4-((1-(3-fluoropropyl)azetidin-3-ylidene)methyl)phenoxy)benzo[b]thiophene-6-carboxylic Acid

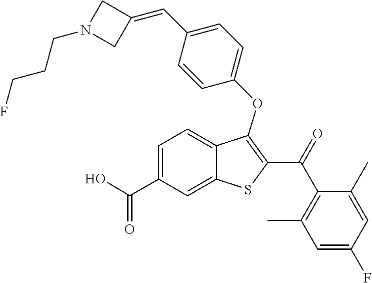 [SMILES: Cc1cc(F)cc(C)c1C(=O)c1sc2cc(C(=O)O)ccc2c1Oc1ccc(C=C2CN(CCCF)C2)cc1]

Example 4 was prepared following a similar procedure to that of step 2 of Example 1 from methyl 2-(4-fluoro-2,6-dimethylbenzoyl)-3-(4-((1-(3-fluoropropyl)azetidin-3-ylidene)methyl)phenoxy)benzo[b]thiophene-6-carboxylate (Intermediate 9) give 44 mg (38%) of 2-(4-fluoro-2,6-dimethylbenzoyl)-3-(4-((1-(3-fluoropropyl)azetidin-3-ylidene)methyl)phenoxy)benzo[b]thiophene-6-carboxylic acid.

The compounds according to table 1 above were subjected to pharmacological tests for determining their degradation effects on estrogen receptors.

### Test: Estrogen Receptor Degradation Activity

Said test involves measuring the in vitro degradation activity of the compounds of the Table 1a.

The measurements of the degradation activities were made using a breast cancer cell ERα in cell western assay as described hereunder.

MCF7 cells (ATCC) were seeded in 384 wells microplate (collagen coated) at a concentration of 10000 cells/30 μL per well in red phenol free MEM alpha medium (invitrogen) containing 5% charcoal dextran striped FBS. The following day, 9 points serial 1:5 dilution of each compound was added to the cells in 2.5 μL at final concentrations ranging from 0.3-0.0000018 μM (in table 2), or 0.1 μM for fulvestrant (using as positive control). At 4 hours post compound addition the cells were fixed by adding 25 μL of formalin (final concentration 5% formalin containing 0.1% triton) for 10 minutes at room temperature and then washed twice with PBS. Then, 50 μL of LI-COR blocking buffer containing 0.1% Triton was added to plate for 30 minutes at room temperature. LI-COR blocking buffer was removed and cells were incubated overnight at cold room with 50 μL anti-ER rabbit monoclonal antibody (Thermo scientific MA1-39540) diluted at 1:1000 in LI-COR blocking buffer containing 0.1% tween-20. Wells which were treated with blocking buffer but no antibody were used as background control. Wells were washed twice with PBS (0.1% tween-20) and incubated at 37° C. for 60 minutes in LI-COR (0.1% tween-20) containing goat anti-rabbit antibody Alexa 488 (1:1000) and Syto-64 a DNA dye (2 μM final concentration). Cells were then washed 3 times in PBS and scanned in ACUMEN explorer (TTP-Labtech). Integrated intensities in the green fluorescence and red fluorescence were measured to determine the levels of ERα and DNA respectively.

The degradation activity with respect to estrogen receptors in this test is given by the concentration which degrades 50% of the estrogen receptor (or IC₅₀) in nM.

The % of ERα levels decrease were determined as follows: % inhibition=100*(1−(sample−fulvestrant: DMSO−fulvestrant)).

The Table 2 below indicates the estrogen receptor degradation activity results for the compounds of Table 1a tested at 0.3 μM, and demonstrates that said compounds have a significant degradation activity on estrogen receptors.

TABLE 2

| | | % |
|---|---|---|
| Compound | Degradation | Degradation |
| No. | IC₅₀ (nM) | At 0.3 μM |
| 1 | 5 | 91 |
| 2 | 5 | 92 |
| 3 | 3 | 85 |
| 4 | 0.3 | 98 |
| 5 | 12 | 91 |
| 6 | 3 | 84 |

It is therefore apparent that the tested compounds have degradation activities for estrogen receptors, with IC50 less than 1 μM and with degradation levels greater than 50%. The compounds of formula (I) can therefore be used for preparing medicaments, especially medicaments which are degraders of estrogen receptors.

Accordingly, also provided herein are medicaments which comprise a compound of the formula (I), or a pharmaceutically acceptable salt thereof.

Herein are also provided the compounds of formula (I) defined above, or pharmaceutically acceptable salts thereof, for use as medicines.

Herein are also provided the compounds of formula (I) defined above, or pharmaceutically acceptable salt thereof, for use in therapy, especially as inhibitors and degraders of estrogen receptors.

Herein are also provided the compounds of formula (I) defined above, or a pharmaceutically acceptable salts thereof, for use in the treatment of ovulatory dysfunction, cancer, endometriosis, osteoporosis, benign prostatic hypertrophy or inflammation.

A particular aspect is a compound of formula (I) defined above, or a pharmaceutically acceptable salt thereof, for use in the treatment of cancer.

In an embodiment, the cancer is a hormone dependent cancer.

In another embodiment, the cancer is an estrogen receptor dependent cancer, particularly the cancer is an estrogen receptor a dependent cancer.

In another embodiment, the cancer is selected from breast, ovarian, endometrial, prostate, uterine, cervical and lung cancer, or a metastasis thereof.

In another embodiment, the metastasis is a cerebral metastasis.

In another embodiment, the cancer is breast cancer. Particularly, the breast cancer is an estrogen receptor positive breast cancer (ERα positive breast cancer).

In another embodiment, the cancer is resistant to anti-hormonal treatment.

In a further embodiment, the compound of formula (I) is as used as single agent or in combination with other agents such as CDK4/6, mTOR or PI3K inhibitors.

According to another aspect, herein is provided a method of treating the pathological conditions indicated above, comprising administering to a subject in need thereof a therapeutically effective amount of a compound of formula (I), or a pharmaceutically acceptable salt thereof. In an embodiment of this method of treatment, the subject is a human.

Herein is also provided the use of a compound of the formula (I), or a pharmaceutically acceptable salt thereof, for the manufacture of a medicament useful in treating any of the pathological conditions indicated above, more particularly useful in treating cancer.

Herein are also provided the pharmaceutical compositions comprising as active principle a compound of formula (I). These pharmaceutical compositions comprise an effective dose of at least one compound of formula (I), or a pharmaceutically acceptable salt thereof, and also at least one pharmaceutically acceptable excipient.

The said excipients are selected, in accordance with the pharmaceutical form and method of administration desired, from the customary excipients, which are known to a person skilled in the art.

In the pharmaceutical compositions for oral, sublingual, subcutaneous, intramuscular, intravenous, topical, local, intra-tracheal, intranasal, transdermal or rectal administration, the active principle of formula (I) above, or its base, acid, zwitterion or salt thereof, may be administered in a unit administration form, in a mixture with conventional pharmaceutical excipients, to animals and to human beings for the treatment of the above disorders or diseases.

The unit administration forms appropriate include oral forms such as tablets, soft or hard gel capsules, powders, granules and oral solutions or suspensions, sublingual, buccal, intra-tracheal, intra-ocular and intra-nasal administration forms, forms for inhalative, topical, transdermal, subcutaneous, intra-muscular or intravenous administration, rectal administration forms and implants. For topical application it is possible to use the compounds of formula (I) in creams, gels, ointments or lotions.

As an example, a unit administration form of a compound of formula (I) in tablet form may comprise the following components:
Compound of formula (I) 50.0 mg
Mannitol 223.75 mg
Sodium croscarmellose 6.0 mg
Corn starch 15.0 mg
Hydroxypropylmethylcellulose 2.25 mg
Magnesium stearate 3.0 mg

There may be particular cases in which higher or lower dosages are appropriate. According to usual practice, the dosage that is appropriate for each patient is determined by the doctor according to the mode of administration and the weight and response of the said patient.

1. A compound of the formula (I) or a pharmaceutically acceptable salt thereof: 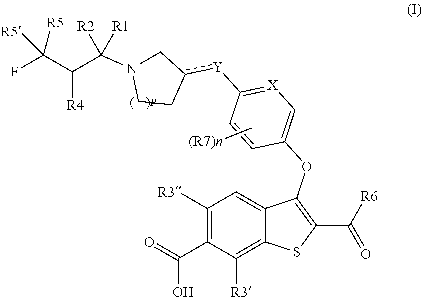
wherein:
R1 and R2 independently represent a hydrogen atom or a deuterium atom;
R3′ and R3″ independently represent a hydrogen atom, a methyl group, a methoxy group, a chlorine atom, a fluorine atom, or a cyano group;
R4 represents a hydrogen atom or a fluorine atom;
R5 and R5′ independently represent a hydrogen atom or a fluorine atom;
Y represents —CH₂—, —CH═, —CR8=, —O— or —NH—, wherein R8 represents a fluorine atom or a (C₁-C₃)alkyl group; 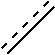
represents a single bond or a double bond;
p is 0 or 1;
X represents —CH═, —N═ or —CR″═, wherein R″ represents a (C₁-C₃)alkyl group or a halogen atom, such as a fluorine or a chlorine atom, a cyano group, or a (C₁-C₃)fluoroalkyl group, such as a trifluoromethyl;
R7 independently represents a (C₁-C₃)alkyl group, such as a methyl group, a halogen atom, such as a fluorine atom, a cyano group, or a (C₁-C₃)fluoroalkyl group, such as a trifluoromethyl;
n is 0, 1 or 2; and
R6 represents a mono or bicyclic ring selected from
a phenyl group,
a 5- or 6-membered monocyclic heteroaryl group, and
a 7-, 8-, 9- or 10-membered bicyclic heterocycle group,
said mono or bicyclic ring being optionally substituted by 1, 2, 3 or 4 groups selected from a halogen atom, a hydroxyl group, a (C₁-C₆)alkyl group optionally substituted with a cyano group or a —OH group, a (C₃-C₆)cycloalkyl group, a (C₁-C₆)fluoroalkyl group, a —CN group, a (C₁-C₆)alkoxy group, a (C₁-C₆) fluoroalkoxy group, a trifluoromethylsulfonyl group, a (C₁-C₄)alkylthio group, a (C₁-C₄)fluoroalkylthio group and a (C₁-C₄)alkylsulfonyl group, provided that the compounds with p=1 and Y is —O— are excluded.

2. The compound of formula (I) according to claim 1, or a pharmaceutically acceptable salt thereof, characterized in that R1 and R2 are a hydrogen atom.

3. The compound of formula (I) according to claim 1 or 2, or a pharmaceutically acceptable salt thereof, characterized in that R3′ and R3″ both represent a hydrogen atom.

4. The compound of formula (I) according to any one of claims 1 to 3, or a pharmaceutically acceptable salt thereof, characterized in that R4 represent a hydrogen atom.

5. The compound of formula (I) according to any one of claims 1 to 4, or a pharmaceutically acceptable salt thereof, characterized in that R5 and R5′ both represent a hydrogen atom.

6. The compound of formula (I) according to any one of claims 1 to 5, or a pharmaceutically acceptable salt thereof, characterized in that Y is —CH₂—, —CH═, —O— or —NH—, in particular Y is —CH₂—, —CH═ or —CR8=.

7. The compound of formula (I) according to any one of claims 1 to 6, or a pharmaceutically acceptable salt thereof, characterized in that n is 0.

8. The compound of formula (I) according to any one of claims 1 to 7, or a pharmaceutically acceptable salt thereof, characterized in that X represents —CH═.

9. The compound of formula (I) according to any one of claims 1 to 8, or a pharmaceutically acceptable salt thereof, characterized in that R6 represents a phenyl group optionally substituted by 1, 2, 3 or 4 groups selected from a halogen atom, and a (C₁-C₆)alkyl group, more particularly optionally substituted by 2, 3 or 4 groups selected from a halogen, for example a fluorine atom, and a methyl group and even more particularly by one fluorine atom and by two methyl groups.

10. The compound of formula (I) according to anyone of claims 1 to 9, or a pharmaceutically acceptable salt thereof, in particular hydrochloride salt thereof, characterized in that said compound is selected from the following compounds:
2-(4-fluoro-2,6-dimethylbenzoyl)-3-(4-((1-(3-fluoropropyl)azetidin-3-yl)amino)phenoxy)benzo[b]thiophene-6-carboxylic acid (1),
2-(4-fluoro-2,6-dimethylbenzoyl)-3-(4-((1-(3-fluoropropyl)azetidin-3-yl)oxy)phenoxy)benzo[b]thiophene-6-carboxylic acid (2),
2-(4-fluoro-2,6-dimethylbenzoyl)-3-(4-((1-(3-fluoropropyl)azetidin-3-yl)methyl)phenoxy)benzo[b]thiophene-6-carboxylic acid (3),
2-(4-fluoro-2,6-dimethylbenzoyl)-3-(4-((1-(3-fluoropropyl)azetidin-3-ylidene)methyl)phenoxy)benzo[b]thiophene-6-carboxylic acid (4),
(S)-2-(4-fluoro-2,6-dimethylbenzoyl)-3-(4-((1-(3-fluoropropyl)pyrrolidin-3-yl)oxy)phenoxy)benzo[b]thiophene-6-carboxylic acid (5), and
(S)-2-(4-fluoro-2,6-dimethylbenzoyl)-3-(4-((1-(3-fluoropropyl)pyrrolidin-3-yl)amino)phenoxy)benzo[b]thiophene-6-carboxylic acid (6).

11. A process for preparing a compound of formula (I) as described in anyone of claims 1 to 10, wherein a compound of formula1D 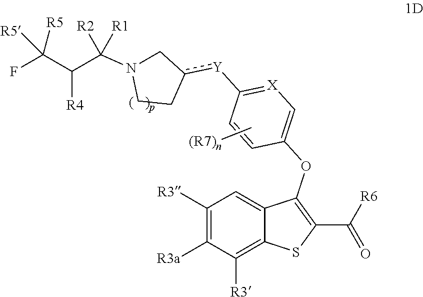
wherein R1, R2, R3′, R3″, R4, R5, R5′, R6, R7, 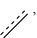
n, p, X and Y are as defined in any of claims 1 to 9 and R3a is a carboxylic ester such as COOMe or COOEt is converted to compound of formula (I), in presence of a source of hydroxide ions, such as NaOH or LiOH in solution in methanol or THF, said step being optionally preceded by a step of obtaining compound 1D, wherein a compound of formula 1F 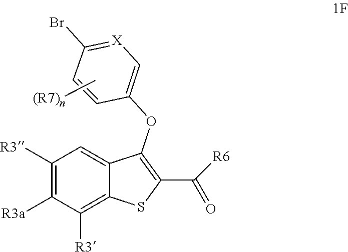
wherein R3′, R3″, R6, R7, n, and X are defined above and R3a is a carboxylic ester such as COOMe or COOEt, is subjected to a coupling step with one of compounds 1G 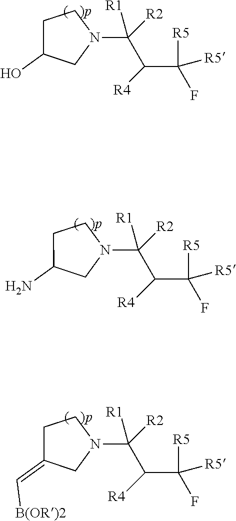
wherein R1, R2, R4, R5, R5′ and p are as defined above.

12. A process for preparing a compound of formula (I) as described in anyone of claims 1 to 10, wherein a compound of formula 1D 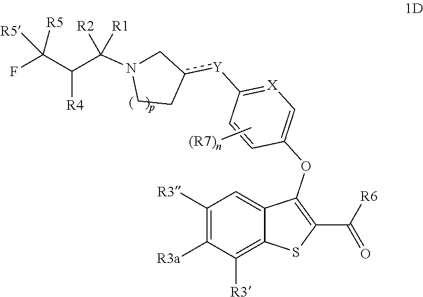
wherein R1, R2, R3′, R3″, R4, R5, R5′, R6, R7, 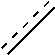
n, p, X and Y are as defined in any of claims 1 to 9 and R3a is a carboxylic ester such as COOMe, COOEt, is converted to compound of formula (I), in presence of a source of hydroxide ions, such as NaOH or LiOH in solution in methanol or THF, said step being optionally preceded by a step of obtaining compound 1D, wherein a compound of formula 1B 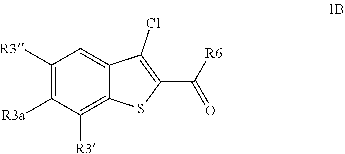
wherein R3′, R3″ and R6 are as defined above and R3a is a carboxylic ester such as COOMe or COOEt, is subjected to a SNAr reaction in a solvent such as DMF, in the presence of a base, with compound 1C 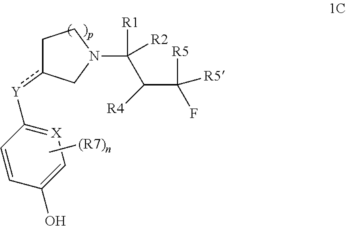
wherein R1, R2, R4, R5, R5′, p, n, R7, X, 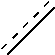
and Y are as defined above.

13. A process for preparing a compound of formula (I) as described in anyone of claims 1 to 10, wherein a compound of formula formula 1D 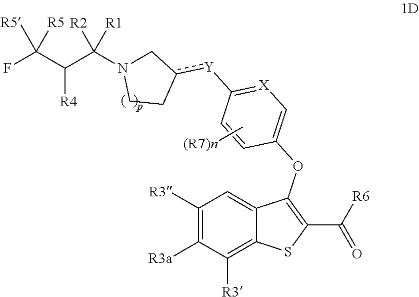
wherein R1, R2, R3′, R3″, R4, R5, R5′, R6, R7, 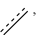
, n, p, X and Y are as defined in any of claims 1 to 9 and R3a is a carboxylic ester such as COOMe or COOEt is converted to compound of formula (I), in presence of a source of hydroxide ions, such as NaOH or LiOH in solution in methanol or THF, said step being optionally preceded by a step of obtaining compound 1D, wherein a compound of formula 1J 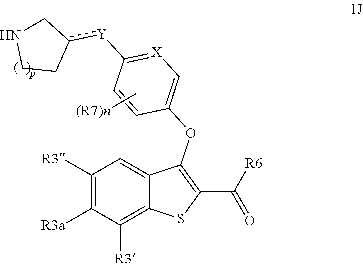
wherein R3′, R3″, Y, p, 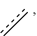
, X, R7, n and R6 are as defined above and R3a is a carboxylic ester such as COOMe or COOEt, is converted in presence of a base with compound 1K 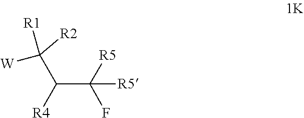
wherein W is Cl, Br or I or OSO₂R with R being CH₃, PhMe, CF₃ or CF₂CF₂CF₂CF₃.

14. Compounds selected from compounds of formula 1D, 1F, 1H and 1L 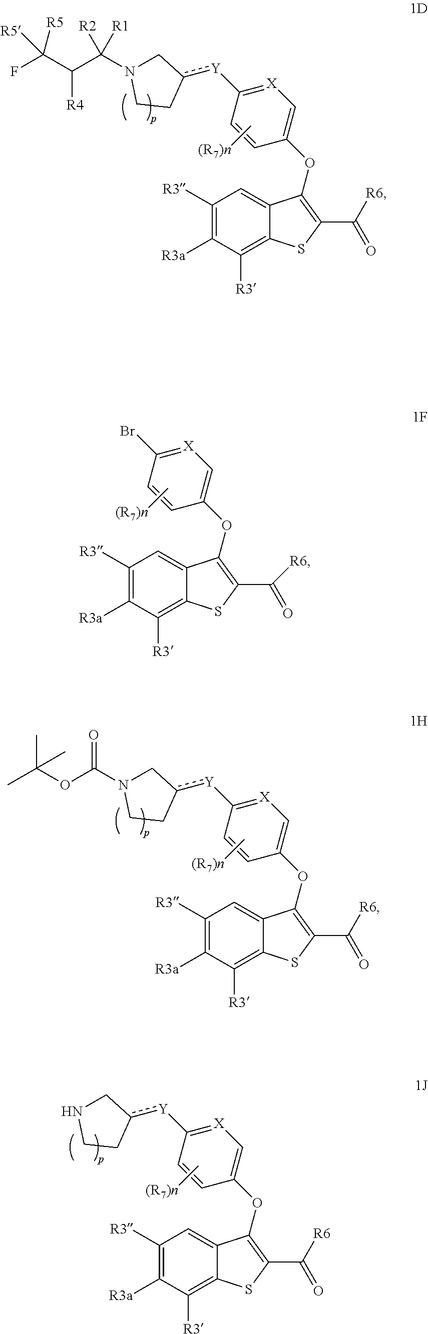
wherein R1, R2, R3′, R3″, R4, R5, R5′, R6, R7, 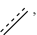
, n, p, X and Y are as defined in any of claims 1 to 9 and R3a is a carboxylic ester such as COOMe or COOEt.

15. A medicament, characterized in that it comprises a compound of formula (I) according to any of claims 1 to 10, or a pharmaceutically acceptable salt thereof.

16. A pharmaceutical composition, characterized in that it comprises a compound of formula (I) according to any of claims 1 to 10, or a pharmaceutically acceptable salt thereof, and at least one pharmaceutically acceptable excipient.

17. A compound of formula (I) according to any of claims 1 to 10, or a pharmaceutically acceptable salt thereof, for use as an inhibitor and degrader of estrogen receptors.

18. A compound of formula (I) according to any of claims 1 to 10, or a pharmaceutically acceptable salt thereof, for use in the treatment of ovulatory dysfunction, cancer, endometriosis, osteoporosis, benign prostatic hypertrophy or inflammation.

19. A compound of formula (I) for use according to claim 18, or a pharmaceutically acceptable salt thereof, for use in the treatment of cancer.
